(19) United States
(12) Patent Application Publication
(10) Pub. No.: US 2026/0091161 A1
(43) Pub. Date: Apr. 2, 2026

(54) THIOL-ENE HYDROGELS

(71) Applicant: ALLARTA LIFE SCIENCE INC., Dundas (CA)

(72) Inventors: Harald Donald Helmut STOVER, Dundas (CA); Samantha ROS, Hamilton (CA); Nicholas BURKE, Dundas (CA); Carl Daniel ELLIS, Ancaster (CA); Scott Brice CAMPBELL, Hamilton (CA); Sarah Alison STEWART, Ancaster (CA); Tobias FUEHRMANN, Toronto (CA); Nadia AL-BANNA, Toronto (CA); Roopali CHAUDHARY, Hamilton (CA); Mitchell JOHNSON, Hamilton (CA); Nicole Aliida MANGIACOTTE, East Aurora, NY (US); Hossein GOLZAR, Burlington, CA (US)

(21) Appl. No.: 19/111,212

(22) PCT Filed: Sep. 19, 2023
(86) PCT No.: PCT/CA2023/051447

§ 371 Date: Mar. 12, 2025

Related U.S. Application Data
(60) Provisional application No. 63/408,049, filed on Sep. 19, 2022.

(51) Int. Cl. A61L 27/52; A61L 27/38; A61L 27/54; B29C 48/16; B33Y 70/00

(52) U.S. Cl. CPC A61L 27/52; A61L 27/38; A61L 27/54; B29C 48/16; A61L 2300/62; A61L 2300/64; B33Y 70/00

(57) ABSTRACT

There is provided a hydrogel string for encapsulating a biological material. The hydrogel string has a thiol-ene crosslinked polymer with a first side-chain functionalized backbone polymer functionalized with an activated alkene crosslinked with a free or protected thiol-containing group present on a second side-chain functionalized backbone polymer. The biological material is encapsulated within the thiol-ene crosslinked polymer.

### CROSS REFERENCE TO A RELATED APPLICATION

This disclosure claims priority of U.S. provisional application No. 63/408,049 filed Sep. 19, 2022, which is incorporated herein by reference in its entirety.

### TECHNICAL FIELD

This disclosure relates to the field of hydrogel polymers, particularly the encapsulation of biological materials such as cells, cell aggregates, tissues, and the like inside the hydrogel polymers.

### BACKGROUND OF THE ART

Synthetic polymers are chemically defined, scalable, and are increasingly being used to form hydrogels, often by employing efficient, biocompatible crosslinking chemistry such as “click reactions”. Synthetic polymer hydrogels show many features reminiscent of natural extracellular matrices (ECM), and are hence being explored for use as ECM mimics. They can provide structural integrity to tissue constructs, control drug delivery, and serve as immuno-isolation barriers for transplantation of therapeutic cells.

Commonly, these reactive polymers are combined with aqueous solutions of sodium alginate containing therapeutic cells or model cells, dropped into calcium or barium containing gelling baths to form calcium or barium alginate beads containing one or both of the mutually reactive polymeric gel formers. Where only one reactive polymer is included within these alginate beads, the second gel former, sometimes referred to as crosslinker, must be introduced to the beads by in-diffusion after beads have been formed.

The alginate chemistry has been thoroughly investigated but there remains to be any alginate based encapsulating material with in vivo therapeutic success. Therefore, improvements in the chemistry of the synthetic polymers and/or of the physical properties of the combined hydrogel are desired.

### SUMMARY

In one aspect there is provided a hydrogel string including a thiol-ene crosslinked polymer comprising a first side-chain functionalized backbone polymer functionalized with an activated alkene crosslinked with a free or protected thiol-containing group present on a second side-chain functionalized backbone polymer, and a biological material encapsulated within the thiol-ene crosslinked polymer. In this context, a hydrogel string refers to a string structure that includes at least one hydrogel polymer. In some embodiments, the hydrogel string has an aspect ratio of at least 5, preferably at least 100. In some embodiments, the hydrogel string comprises alginate, hyaluronic acid, gelatin, hydroxypropylcellulose, carboxymethylcellulose, methylcellulose or elastin. In some embodiments, the biological material is a cell, a cell aggregate, or cell spheroid, and the hydrogel string optionally further encapsulates angiogenic and/or chemotactic agents. In some embodiments, the activated alkene is vinylsulfone, maleimide, acrylate or a methacrylate. In some embodiments, the thiol-containing group is 2-pyridinethiol or cystamine. In some embodiments, the backbone polymer is a homopolymer of polyacrylic acid, a homopolymer of polymethacrylic acid, or copolymers of acrylic acid and methacrylic acid. In some embodiments, the hydrogel string further includes a capping agent on the surface of the hydrogel string neutralizing vinyl sulfone groups. In some embodiments, the hydrogel string has an outer diameter of less than 2000 μm, preferably less than 1000 μm and more preferably less than 600 μm. In some embodiments, the thiol-ene crosslinked polymer forms an outer shell encapsulating a core of the biological material. In some embodiments, the shell has a heterogeneous density, wherein an outer surface has a higher density than an inner surface as measured by fluorescence microscopy.

In one aspect, there is provided a three dimensional hydrogel structure formed by interconnected hydrogel strings as defined herein including a plurality of the thiol-ene crosslinked polymers forming at least a portion of the interconnected hydrogel strings wherein each of the hydrogel strings is connected by thiol-ene crosslinks forming a continuous crosslinked structure. In some embodiments, the three dimensional hydrogel structure is a patch formed by 3D printing of the hydrogel strings into shapes, wherein the hydrogel strings or portions thereof intersect or cross over each other to form a microporous 2-dimensional array, preferably designed to maximize surface area needed for metabolic exchange of the therapeutic cells, preferably wherein the biomaterial can migrate between intersecting strings or portions thereof.

In one aspect, there is provided a process of producing a hydrogel string comprising: continuously extruding or co-extruding a first polymer containing free or protected thiol groups and a second polymer containing vinyl groups into a bath, preferably an aqueous bath, containing a reactive agent to drive the gelation of the first and second polymers; and allowing a crosslinking reaction between the thiol groups and the vinyl groups to occur. In a further aspect, there is provided a process of producing interconnected hydrogel strings, comprising: extruding or co-extruding a composition comprising a first polymer containing free or protected thiol groups and a second polymer containing vinyl groups into a bath, preferably an aqueous bath, containing a reactive agent to drive the gelation of the first and second polymers, and wherein the bath has a low concentration of reactive agent, a low concentration of reactive agent being a level that enables partial cross-linking of the thiol-ene polymer to form a plurality of polymer strings; forming a desired shape with the plurality of polymer strings; and exposing the plurality of polymer strings to a reducing agent to further crosslink the plurality of polymer strings to form the interconnected hydrogel strings. In some embodiments, the extruding or co-extruding further comprises extruding or co-extruding a biocomptible polymer selected from the group consisting of alginate, hyaluronic acid, gelatin, hydroxypropylcellulose, carboxymethylcellulose, methylcellulose, or elastin with the first and second polymers. In some embodiments, the biocompatible polymer is alginate and the bath comprises ions selected from the group consisting of calcium, barium, strontium, copper, zinc, manganese, cobalt, lead, iron, or aluminum, preferably the ions are present in a concentration of 5 mM to 100 mM. In some embodiments, the reactive agent is a reducing agent, preferably tris (2-carboxyethyl) phosphine (TCEP) or tris (hydroxypropyl) phosphine (THPP), and preferably the reactive agent is present in a concentration of 5 mM to 100 mM. In some embodiments, the process further comprises providing a capping agent introduced as the final process step prior to final wash, to convert residual vinyl sulfone groups into more biocompatible groups. “Biocompatible polymers” as used herein refers to polymers that do not induce an immune response in vivo and, in particular, when implanted in a mammal, that substantially interferes with viability and function of encapsulated cells. The biocompatible polymers can also be characterized by low fibrosis such as a PCO score of 0-50%, preferably 0-25%, or more preferably 1-2%. In some embodiments, the ratio of thiol groups to vinyl (ene) groups is from 0.95:1.05 to 0.65:1.35, preferably from 0.9:1.1 to 0.5:1.5, and most preferably 0.8:1.2 to 0.6:1.4. In some embodiments, the process further comprises extruding or co-extruding the polymers into the bath through a needle, preferably a blunt end syringe. In some embodiments, the thiol groups of the first polymer are protected thiol groups, and preferably the first polymer is poly(methylvinylether-alt-maleic anhydride) carrying the protected thiol groups.

In one aspect there is provided a transplant comprising at least one hydrogel string as defined in any one of claims 1 to 11 and optionally a supporting substrate, wherein the at least one hydrogel string is substantially retrievable from an implant site in a live mammalian body after a transplantation time of six weeks to one year, or longer such as 2-3 years. The term “substantially retrievable” as used herein means that the hydrogel strings are retrieved but a portion of the string may have degraded or decomposed by the time retrieval happens. Moreover, a portion of the strings may remain in the body and not be retrieved however this portion is small and does not lead to any long term effects on the subject having received the transplant. In one embodiment, at least 90% of the hydrogel strings are retrieved. There is also provided a method of transplanting the transplantable device as described, comprising: depositing at least one hydrogel string containing the biological material onto omental tissue of a subject in need thereof, and folding over and securing the omental tissue by either stitching or using tissue glue which is preferably fibrinogen and/or thrombin glues. The method may be an open surgery or a laparoscopic procedure.

In a further aspect, there is provided a hydrogel comprising a first side-chain functionalized backbone polymer functionalized with an activated alkene crosslinked with a cystamine-containing group present on a second side-chain functionalized backbone polymer. In some embodiments, the hydrogel further comprises alginate, hyaluronic acid, gelatin, hydroxypropylcellulose, carboxymethylcellulose, methylcellulose, or elastin. In some embodiments, the hydrogel encapsulates a biological material, preferably a cell, a cell aggregate, or a cell spheroid, of either mammalian or bacterial origin. In some embodiments, the activated alkene is vinylsulfone, acrylate or a methacrylate. In some embodiments, the backbone polymer is a homopolymer of polyacrylic acid, a homopolymer of polymethacrylic acid, or copolymers of acrylic acid and methacrylic acid. In some embodiments, the cystamine containing group has a poly(methyl vinyl ether-alt-maleic anhydride) backbone polymer. In some embodiments, the hydrogel further comprises a capping agent on the surface of the hydrogel string neutralizing vinyl sulfone groups. In some embodiments, the hydrogel is a capsule, a string or a patch. In some embodiments, the hydrogel is crosslinked.

In an additional aspect, there is provided a kit for encapsulating a biological material such as cells, including: a solution containing 5 to 35 mol. % of a polymer containing free or protected thiol groups; a solution containing 5 to 35 mol. % of a polymer containing vinyl groups; alginate in a buffer solution in a concentration of 1 to 4 wt. % adapted to receive a biological material to be encapsulated; optionally, a calcium or barium salt to form an ionic gelation bath; and optionally, a reducing agent for the ionic bath or for post-printing gelation of the thiol-ene polymers. The kit can be used for producing a hydrogel string as defined herein, a three dimensional hydrogel structure as defined herein, a transplant as defined herein, or a hydrogel as defined herein.

Many further features and combinations thereof concerning the present improvements will appear to those skilled in the art following a reading of the instant disclosure.

### DESCRIPTION OF THE DRAWINGS

FIG. 1 is a graph showing the detailed fibrosis scoring (pericapsular overgrowth-PCO) of two mice (Y1-1 and Y1-2) having received capsules formed by poly(methyl vinyl ether-alt-maleic anhydride) (PMM) with a protected thiol (PMM-Spy) and pendant vinyl sulfone (PMM-VS) combined in a ratio of 0.9:1.1. Capsules with 0-25% of their surface coated with PCO are binned into group (A), capsules with 25-50% PCO into group (B), capsules with 50-75% PCO into group (C) and capsules with 75-100% PCO into group (D), as per Example 2.

FIG. 2A is a graph showing the insulin level in vitro in the conditions of A: free islets with 2.8 mM glucose stimulation, B: free islets with 28 mM stimulation, C: islets encapsulated in thiol-ene crosslinked cysteine coated (TEC) polymer capsules with 2.8 mM stimulation, D: islets encapsulated in TEC polymer capsules with 28 mM stimulation, E: islets encapsulated in TEC polymer capsules with 2.8 mM stimulation (duplicate), and F: islets encapsulated in TEC capsules with 28 mM stimulation (duplicate), as per Example 3.

FIG. 2B is a graph showing the blood glucose (BG) level over time for STZ-diabetic C57BL/6j mice with either 900 islet equivalents (IEQ) of human donor islets encapsulated in TEC capsules as formed in FIG. 2A, or blank capsules without islets, labelled on the figure respectively as “islets” and “blanks”, as per Example 3.

FIG. 2C is a graph showing the concentration of human C-peptide for mice having received the encapsulated islets the “blanks” or “islets” of FIG. 2B. They show human C-peptide in their circulation at levels that are higher than control mice (i.e., blanks), as per Example 3.

FIG. 3A is a fluorescence image of a calcium alginate (CA) TEC string having a length of 30 cm and made with 1.2% alginate, and 1.5% 0.7:1.3 TEC, as per Example 4.

FIG. 3B is an image of 1.2% alginate, 1.5% 0.7:1.3 TEC string loaded with ˜61 IEQ/cm of rat islet clusters, as per Example 4.

FIG. 3C is an image of 1.2% alginate, 1.5% 0.7:1.3 TEC string loaded with ˜61 IEQ/cm of rat islet clusters, as per Example 4.

FIG. 3D is a graph showing the average blood glucose concentration after 11 days post-implantation of 15 cm 1.2% alginate, 1.5% 0.7:1.3 TEC string segments loaded with ˜61 IEQ/cm of rat islet clusters (▪) versus 15 cm blank 1.2% alginate, 1.5% 0.7:1.3 TEC string segments in C57BL6 mice (•) (n=4), as per Example 4.

FIG. 3E shows intact 15 cm blank 1.2% alginate, 1.5% 0.7:1.3 TEC string segments after 14 days of implantation in C57BL6 mice, as per Example 4.

FIG. 3F shows broken 15 cm blank 1.2% alginate, 1.5% 0.7:1.3 TEC string segments after 14 days of implantation in C57BL6 mice, as per Example 4.

FIG. 4 is a schematic of a post-bioprinting processing to obtain a 4-layer alginate patch, as per Example 4.

FIG. 5A is a microscopy image of 1.0% CA-TE Cys30 polymer string section (scale bar 500 μm), as per Example 4.

FIG. 5B is a microscopy image of 1.0% CA-TE Cys30 polymer string encapsulating an islet (scale bar 500 μm), as per Example 4.

FIG. 5C is a microscopy image of 1.5% CA-TE polymer string before citrate treatment (scale bar 500 μm), as per Example 4.

FIG. 5D is a microscopy image of 1.5% CA-TE polymer string after 1 min exposure to 70 mmol citrate (scale bar 500 μm), as per Example 4.

FIG. 5E is a microscopy image of 1.5% CA-TE polymer string after 10 min exposure to 70 mmol citrate (scale bar 500 μm), as per Example 4.

FIG. 5F is a microscopy image of 1.5% CA-TE polymer string after 45 min exposure to 70 mmol citrate (scale bar 500 μm), as per Example 4.

FIG. 5G is a microscopy image of 1.0% CA-TE polymer string before citrate treatment (scale bar 500 μm), as per Example 4.

FIG. 5H is a microscopy image of 1.0% CA-TE polymer string 1 min after exposure to 70 mmol citrate (scale bar 500 μm), as per Example 4.

FIG. 5I is a microscopy image of 1.0% CA-TE polymer string 10 min after exposure to 70 mmol citrate (scale bar 500 μm), as per Example 4.

FIG. 5J is a microscopy image of 1.0% CA-TE polymer string 45 min after exposure to 70 mmol citrate (scale bar 500 μm), as per Example 4.

FIG. 5K is a microscopy image of 0.75% CA-TE polymer string before citrate treatment (scale bar 500 μm), as per Example 4.

FIG. 5L is a microscopy image of 0.75% CA-TE polymer string 1 min after exposure to 70 mmol citrate (scale bar 500 μm), as per Example 4.

FIG. 5M is a microscopy image of 0.75% CA-TE polymer string 45 min after exposure to 70 mmol citrate (scale bar 500 μm), as per Example 4.

FIG. 5N is a photograph of a 0.75% CA-TE polymer string before citrate treatment, as per Example 4.

FIG. 5O is a photograph of a 1.0% CA-TE polymer string before citrate treatment, as per Example 4.

FIG. 5P is a photograph of a 1.5% CA-TE polymer string before citrate treatment, as per Example 4.

FIG. 5Q is a photograph of a 0.75% CA-TE polymer string after citrate treatment, as per Example 4.

FIG. 5R is a photograph of a 1.0% CA-TE polymer string after citrate treatment, as per Example 4.

FIG. 5S is a photograph of a 1.5% CA-TE polymer string after citrate treatment, as per Example 4.

FIG. 5T is a LIVE/DEAD fluorescently stained image of encapsulated islets in a TEC string before citrate treatment, as per Example 4.

FIG. 5U is a LIVE/DEAD fluorescently image of encapsulated islets in a TEC string after citrate treatment, as per Example 4.

FIG. 5V is an image of a 30 cm 1.0% CA-TE string (treated with 30 mM cysteine) before being transplanted in vivo in a healthy mouse, as per Example 4.

FIG. 5W is an image of the explanted string of FIG. 5W (25 cm section), as per Example 4.

FIG. 6A is a photograph of a 1% calcium alginate CA-TE string treated with 30 mM cysteine and encapsulating islets at 1 islet/inch (0.4 islets/cm), as per Example 4.

FIG. 6B is a photograph of a 2% calcium alginate CA-TE string treated with 60 mM cysteine and encapsulating islets at 2 islets/inch (0.8 islets/cm), as per Example 4.

FIG. 6C is a microscopy image of 1% CA-TE string (treated with cysteine 30 mM) made from a solution containing 13,600 IEQ/mL (6.8×) that yielded a string with 37 IEQ/cm, as per Example 4.

FIG. 6D is a photograph of the string of FIG. 6C.

FIG. 6E is a microscopy image of 1% CA-TE (treated with cysteine 30 mM) string showing a diameter of 685±18 μm (n=10), as per Example 4.

FIG. 6F is a microscopy image of 2% CA-TE (treated with cysteine 60 mM) string showing a diameter of 722±12 μm (n=10), as per Example 4.

FIG. 6G is a live/dead staining image of rat islets 4 days after encapsulation in a 1% calcium alginate CA-TE string treated with 30 mM cysteine, as per Example 4.

FIG. 6H is a close up of the image of FIG. 6G.

FIG. 7A is a phase contrast microscopy image of 1% TEC Cys 30 mM string, as per Example 4.

FIG. 7B is a bright field contrast microscopy image of 1% TEC Cys 30 mM string, as per Example 4.

FIG. 7C is a bright field microscopy image of 1% TEC Cys 30 mM showing the end of the string, as per Example 4.

FIG. 8 is a bar graph showing the average insulin concentration produced by human islets encapsulated in TEC-cystamine strings or islets at days 0 and 4, as per Example 7.

FIG. 9A is a microscopy image of a pancreatic islet encapsulated in a TEC-cystamine string, as per Example 7.

FIG. 9B is a microscopy image of a pancreatic islet encapsulated in a TEC-cystamine capsule, as per Example 7.

FIG. 10A is a bar graph showing the average insulin concentration produced by rat islets encapsulated in TEC-cystamine string as per Example 7, after 14 days post-transplantation (conditions labeled Ahr-1, Ahr-3) and 42 days post-transplantation (conditions labeled Ahr-4, Ahr-5 and Ahr-6).

FIG. 10B is a bar graph showing the average insulin release by rat islets encapsulated in TEC-cystamine string as per Example 7, 42 days post-transplantation.

FIG. 11A is a microscopy image showing live/dead staining of an encapsulated islet in a TEC-cystamine bead, as per Example 7.

FIG. 11B is a microscopy image of live/dead staining comparing an encapsulated islet in a TEC-cystamine bead and in a TEC-cystamine string, as per Example 7.

FIG. 12 is a graph showing the variation of blood glucose (BG) over time for blank TEC-cystamine strings and capsules transplanted, free islets transplanted and TEC-cystamine strings and capsules encapsulating islets transplanted as per Example 7.

FIG. 13 is a bar graph showing the average insulin concentration in four different animals labeled (S35-5 to S35-8) at times 0, 1 h, 2 h, and 3 h transplanted with TEC-cystamine capsules encapsulating human islets after 29 days, as per Example 7.

FIG. 14A is a live/dead staining of an encapsulated islet in TEC-cystamine capsules after 29 days of transplantation, as per Example 7.

FIG. 14B is a live/dead staining of a second encapsulated islet in TEC-cystamine capsules after 29 days of transplantation, as per Example 7.

FIG. 14C is a live/dead staining of a third encapsulated islet in TEC-cystamine capsules after 29 days of transplantation, as per Example 7.

FIG. 14D is a close up on the islet of FIG. 14A.

FIG. 14E is a close up on the islet of FIG. 14B.

FIG. 14F is a close up on the islet of FIG. 14C.

FIG. 15 is a photograph of a gel patch made of three layers of TEC-cystamine strings (no materials were encapsulated), as per Example 7.

FIG. 16A is a live/dead staining of encapsulated islets in a gel patch made of TEC-cystamine strings with 2 wt. % alginate (500 μm scale bar), as per Example 8.

FIG. 16B is a wider view of the gel patch of FIG. 16A (scale bar 2000 μm).

### DETAILED DESCRIPTION

### Thiol-Ene (TE) Polymer Hydrogels

Thiol-ene (TE) polymers are a pair of hydrophilic polymers, one functionalized with an activated alkene group and the other with a free or protected thiol such as SPy, that when combined may form a crosslinked hydrogel. The thiol/ene polymer pair may form hydogels alone or they may provide enhanced robustness by forming a reinforcing network within a primary hydrogel gel such as calcium or barium alginate. This network is formed by a thiol-ene addition reaction that can be triggered either in the gelling or printing bath, or during a separate subsequent step, by exposing for example a SPy-protected polymeric thiol component to a reducing agent such as the mild reducing agent tris (2-carboxyethyl) phosphine (TCEP) or tris(hydroxypropyl) phosphine (THPP), which liberate free thiol groups on the thiol-functional vinyl polymer component (e.g., poly(methylvinylether-alt-maleic anhydride) (PMM)). For example, once generated from a protected thiol precursor such as SPy, the free pendant thiol groups couple spontaneously within seconds to minutes with the vinylsulfone groups on the vinylsulfone-functional PMM, leading to a covalently crosslinked PMM network distributed throughout the calcium alginate hydrogel. In some embodiments, the thiol-ene polymer network is defined as a covalently crosslinked polymer network with a S atom covalently linking the thiol component to one of the carbon atoms that used to be part of a vinyl group (the thiol-ene covalent bond). The formation of a thiol-ene covalent bond is the gelation or curing mechanism between a thiol containing polymer and a vinyl containing polymer. In some embodiments, one of or both of the thiol containing polymer and the vinyl containing polymer are oxygen containing polymers (for example having at least two carbonyl groups in each repeat unit). The thiol groups can be free or protected thiol groups. This gelation or curing process, and the formed thiol-ene polymer network, are designed to be compatible with an aqueous environment, and with the presence of viable cells including mammalian cells and bacterial cells. These mammalian cells are usually chosen for their therapeutic action, and may include mammalian cells expressing hormones or enzymes such as but not limited to, insulin, and blood clotting factors, or bacterial cells such as but not limited to, natural or genetically modified Streptococcus thermophilus that are of benefit for disorders of the intestinal tract. These networks retain the highly hydrophilic nature of the gel-forming thiol and ene polymer components. The encapsulation of lactose producing bacteria and other microbiome beneficial bacteria are contemplated herein. For example, a subject in need therefore can receive an oral administration of encapsulated bacteria that are encapsulated in any of the systems described herein including the capsules, patches and strings.

Thiol-ene is an advantageous gelation chemistry because it provides a covalently crosslinked gel unlike chemistries such as pure ionic gelation, or temperature-based gelation (e.g., gelatin and hydrolyzed collagen) that utilize more easily disrupted physical crosslinks. Thiol-ene crosslinking does not require potentially toxic catalysts as used in gelation chemistries such as copper-catalyzed azide/alkyne “click” chemistry. In addition, the thiol-ene polymers can be prepared by fairly simple methods, and they retain their hydrophilicity, unlike the polymers required for strain-promoted azide/alkyne click reactions. The covalently cured polymer networks have the ability to retain their form and integrity under in vivo conditions long term. This is in contrast to some erodable ionically crosslinked networks, including calcium alginate alone. The permanently crosslinked nature of the networks can prevent cell egress from the capsules, filaments or strings, and penetration of immune cells into the network. The TE polymer is preferably combined with another hydrogel such as alginate, elastin, hyaluronic acid, hydroxypropylcellulose, carboxymethylcellulose, methylcellulose or gelatin. In some embodiments, the TE polymer makes up more than 50%, more than 60%, more than 70%, more than 80%, more than 90%, more than 95% or more than 99% by weight of the polymeric scaffold. In some embodiments, the polymeric scaffold consists essentially of or consists of alginate and TE. In other embodiments, the polymeric scaffold consists essentially of or consists of TE. In other embodiments, the polymeric scaffold is formed by a TE polymer and alginate for example in a weight ratio of from 1:10 to 10:1, 1:5 to 5:1, 1:4 to 4:1, 1:3, to 3:1, or 1:2 to 2:1. Moreover, in some embodiments, the hydrogel is further combined with higher molecular weight (MW) PMM polyampholyte (non-reactive) as processing aid/filler. The MW of PMM can be varied via polymerization and/or oxidation, to bridge between 80 kDa and 1 MDa.

Alginate hydrogels can be polymerized by ionic crosslinking, most commonly with calcium ions. However, other ions can be used instead of calcium such as barium, strontium, copper, zinc, manganese, cobalt, lead, iron, or aluminum. Alginate hydrogel can be formed in concentration from 0.5 to 8 wt. %, 0.5 to 6 wt. %, 0.5 to 4 wt. %, 0.5 to 3 wt. % or 0.5 to 2 wt. %. Elastin or hyaluronic acid can for example be used in similar concentration ranges as alginate.

In preferred embodiments, the hydrogel is formed by a thiol-ene polymer and by alginate, and optionally hyaluronic acid. Hyaluronic acid is a non-fibrotic implant additive that can improve the strength and lubricity of the hydrogels. The gelation of both network formers (alginate, thiol/ene) can happen simultaneously upon emergence of the gel forming solution into a gelling bath containing cations (e.g., calcium) and a reducing agent (e.g., TCEP). Optionally, gelation of the thiol/ene network component can be carried out as part of a separate, subsequent exposure of the initially formed calcium alginate gel to a separate TCEP gelling bath. Both networks reinforce each other, such that the covalent network sustains physical integrity for long periods of incubation and implantation. When present, the optional hyaluronic acid is physically trapped within the covalent network and helps provide hydrophilicity.

The hydrophilic backbone of TE polymers may be biocompatible polymers having a molecular weight between about 2000 to 2,000,000 Daltons, such as 5000 to 1,000,000 Daltons, or 20,000 to 500,000 Daltons. One polymer is a polyfunctional Michael acceptor suitable for crosslinking with a second polymer that is a polyfunctional Michael donor. Examples of suitable backbone polymers include, but are not limited to, homopolymers of polyacrylic acid or polymethacrylic acid or copolymers of acrylic acid or methacrylic acid with anionic, uncharged or cationic monomers including but not limited to, styrene sulfonic acid; acrylamides and methacrylamides such as acrylamide and N,N-dimethylacrylamide, or polyethylene glycol (PEG) acrylates and PEG methacrylates with degrees of polymerization of the PEG side chain of 1 to 20 or higher, and N,N-dimethylaminoethylmethacrylamide, N,N-dimethylaminoethyl methacrylate or acrylic anhydride. Other examples include polymers formed by copolymerization of alkyl vinyl ethers with an anhydride, e.g. of a dicarboxylic acid such as maleic, itaconic, or citraconic acid, wherein the alkyl groups consist of ethyl, n-propyl, i-propyl, n, sec or t-butyl groups, and higher alkyl groups (e.g. C5-C12); vinyl ethers of ethylene oxide oligomers with an anhydride (e.g. maleic or itaconic anhydride); polyanhydrides based on polyacrylic acid that was dehydrated to form cyclic anhydride moieties along the polymer backbone; polymers of carboxylic acid anhydrides such as acrylic anhydride, and copolymers of aromatic monomers such as styrene with maleic anhydride or other vinyl-functional anhydrides such as itaconic or citraconic anhydride, e.g. the alternating copolymer of styrene with a vinyl-functional anhydride. Additional backbone polymers include polymers containing epoxy groups such as glycidyl methacrylate, together with optionally neutral or anionic monomers selected from acrylamide and methacrylamide, N-alkylsubstituted acrylamides and methacrylamides, hydroxyethylacrylamide, hydroxyethylmethacrylamide, PEG methacrylates; and acrylic acid, methacrylic acid, vinylbenzenesulfonic acid and their respective alkali metal salts.

In some embodiments, the backbone polymer is a copolymer of an alkyl vinyl ether with an acid anhydride such as maleic, itaconic, or citraconic anhydride such as poly(methyl vinyl ether-alt-maleic acid) since the anhydride groups are readily functionalized and rendered biocompatible by hydrolysis of remaining anhydride moieties following functionalization.

The ene-polymer is a backbone polymer side-chain functionalized with a cross-linkable compound that is reactive with the backbone polymer and comprises an activated alkene functionality. The activated alkene functionality may be, but is not limited to, a vinylsulfone, acrylate, methacrylate, maleimide, or alkynyl group, substituted with electron-withdrawing groups such as esters.

The thiol-polymer is a backbone polymer end- or side-chain functionalized with a cross-linkable compound that comprises a free or protected thiol functionality. The thiol functionality may be, but is not limited to, a thiol or a disulfide such as a 2-pyridinethiol (SPy)-protected thiol.

In one embodiment, the cross-linked hydrogel is formed by reacting an aqueous solution of about 0.5 to 15 wt. % of the side-chain functionalized backbone polymer, and preferably an amount of about 2.5 to 7.5 wt. % of the backbone polymer, with an aqueous solution of a di- or poly-thiol crosslinker. Examples of suitable crosslinkers include polar, water-soluble compounds carrying two or more thiol groups, such as polyethylene glycol (PEG)-dithiols having a molecular weight in the range of from about 200 to 1,000,000 Daltons, preferably a molecular weight of between about 1000 and 20,000 Daltons. The crosslinking reaction involves a Michael Addition of an electron-rich nucleophile (thiol) with an electron-poor alkene (e.g., vinylsulfone, acrylate, maleimide) of the side-chain functionalized backbone polymer in molar ratios ranging from 1:4 to 4:1, and preferably, 1:2 to 2:1. This addition reaction proceeds rapidly under physiological conditions without the need for catalysts and without producing cytotoxic side products.

Theory predicts that an equimolar (1:1) ratio of thiol and ene should result in maximal crosslinking. It was presently found that despite the use of the ideal 1:1 molar ratio of thiol to ene groups, a certain percentage of residual reactive groups (e.g., vinyl) remain on the network, physically restricted from engaging in crosslinking. These residual groups pose risks of subsequent reaction with, and hence immobilization of, undesirable biomolecules such as proteins during encapsulation, and implantation—reactions that could contribute to eliciting an immune response after implantation.

Vinylsulfone in particular is a strongly activated electrophile, able to react with mobile thiol-functional and amine-functional groups on, e.g., proteins present in encapsulation solutions or in serum after implantation. Such attached proteins are known to adopt unnatural conformations compared to circulating immune cells proteins. Pendant vinylsulfone groups on polymer backbones deactivate by spontaneous hydration to form 2-hydroxyethylsulfone groups, a hydrophilic and largely benign group, the half-life of this addition of water to vinylsulfone is on the order of 2-3 days, too long to ensure complete conversion of residual vinylsulfone at time of implantation (usually within 4 to 48 hrs of encapsulation).

To palliate the problem of residual groups, it was surprisingly found that the thiol:ene ratio can be deviated in favor of the ene groups to reduce or eliminate the formation of residual groups, particularly free thiol groups. For example, a ratio of thiol:ene of 0.95:1.05 to 0.5:1.5, preferably 0.95:1.05 to 0.65:1.35, or more preferably from 0.9:1.1 to 0.7 to 1.3. Alternatively, or in addition, the residual groups can be neutralized using a capping agent to obtain a modified residual group. For example, post-functionalization can include deactivation of residual ene groups by addition of deactivating moieties such as cysteine or cysteamine or other thiols to deactivate the reactive alkene sites.

In another embodiment, the cross-linked hydrogel is formed by reacting the side-chain functionalized backbone polymer comprising an activated alkene functionality (i.e., the first side-chain functionalized backbone polymer) with a second side-chain functionalized backbone polymer. The second side-chain functionalized backbone polymer is functionalized with a cross-linkable compound that is reactive with the crosslinkable group (e.g., vinylsulfone) attached to the first backbone polymer and comprises a protected thiol. The backbone polymer of the second side-chain functionalized backbone polymer may be as described above, and may be the same or different than the backbone polymer of the first side-chain functionalized backbone polymer. A preferred backbone polymer is a copolymer of an alkyl vinyl ether with an acid anhydride such as maleic, itaconic, or citraconic anhydride such as poly(methyl vinyl ether-alt-maleic anhydride).

The crosslinkable compound for preparation of the second side-chain functionalized backbone polymer will incorporate an entity that is reactive with the backbone polymer as in the first side-chain functionalized backbone polymer such as an amine group. The crosslinkable compound will also incorporate a protected thiol group. The protected thiol group is not particularly restricted, and may be any group that may be readily deprotected to yield a thiol that will react with the reactive alkene of the first side-chain functionalized backbone polymer to form a covalent linkage between the first and second functionalized backbone polymers. Examples of protected thiol groups that may be incorporated in the crosslinkable compound include, but are not limited to, disulfides, thiopyridines, dithiocarbonates, dithiocarbamates, and thioesters. A preferred crosslinkable compound is S-(2-aminoethylthio)-2-thiopyridine. Another preferred crosslinkable compound is cystamine.

Cystamine incorporates a disulfide group that represents a latent or protected thiol group which can be activated by the reducing agents described above and then serve to crosslink with the other reactive polymer such as the vinyl-sulfone modified polymer. At the same time, the cystamine group is considered more hydrophilic compared with, for example the SPy group, and is hence considered to be more cytocompatible and less likely to attract protein deposition in vivo that might elicit a further immune response. Advantageously, reduction of cystamine functional groups generates reactive thiol groups without the production of hydrophobic small molecule by-products.

The thiol/ene network also offers opportunity for attaching a range of modifying molecules through reaction with the original anhydride groups in case of amine-functional molecules, as well as with residual thiol and/or ene groups, as appropriate. Such molecules could include attachment motifs, such as RGD, cationic groups including primary or secondary amines, e.g., such as dimethylamino alkyl amine (alkyl C2-C5 such as dimethylamino propylamine or dimethylamino ethylamine), neutral groups with thiol moieties or other nucleophiles such as alcohols, e.g., aminoethanol; functional biomolecules such as anti-inflammatory cytokines, cell-promoting proteins, and growth factors, or small molecules (therapeutic agents, e.g., anti-inflammatory agents; detectable labels such as fluorescent labels, e.g., fluoresceinamine, TAMRA-cadaverine, fluorescein cadaverine or rhodamine cadaverine, and the like).

### Effect of Thiol/Ene Components on Need for Alginate

The addition of TE polymers offers an opportunity to reduce the concentration of sodium alginate in the polymeric scaffold as the alginate/thiol-ene polymer mixture has an increased viscosity compared to alginate alone, which aids bead or string formation. In addition, the covalently crosslinked TE network formed within the CA gel means that the bead or string will retain its integrity. Accordingly, in some embodiments, the present disclosure can efficiently employ a sodium alginate concentration of less than 1 wt. % in the precursor solution. In the art, the concentration of 1 wt. % alginate is considered the lower limit for most applications of alginate hydrogels particularly in vivo. Calcium alginate gels formed traditionally from 1 wt. % sodium alginate without any coating or thiol/ene reinforcement are fairly non-fibrotic, and may even disappear in vivo. Thanks to the thiol-ene crosslinked polymers reinforcing the alginate, robust hydrogel strings or beads containing much less than 1 wt. % alginate may be formed as the alginate in the generated strings or patches is lost via partial liquefaction and/or partial extraction post gelation. In one example, citrate can be used to extract alginate post extrusion and/or post gelation. The concentration of alginate can thus be less than 1 wt. % alginate, less than 0.9 wt. %, less than 0.8 wt. %, less than 0.75 wt. %, less than 0.7 wt. %, less than 0.6 wt. % or less than 0.5 wt. %. The advantage of reducing the alginate to less than 1 wt. % is a decrease in the possibility of eliciting a foreign body reaction through immunogenic motifs inherent in alginate itself, or through residual bacterial shell fragment or other proteinaceous contamination not completely removed during the purification of this biomaterial sourced from oceanic locations.

The hydrogels presently prepared do not rely entirely on the ionic crosslinking of sodium alginate with divalent ions such as calcium, strontium, or barium to provide long-term mechanical integrity to the hydrogel matrices. Instead, the presence of sodium alginate helps by:
a) acting as a processing aid, especially to help protect cells from shear forces created during the air-shearing and extrusion processes, and by
b) maintaining the shape of the air-sheared/extruded gels immediately upon entering the gelling bath, and until the covalent crosslinking has taken place.

Subsequent to covalent crosslinking, the ionically gelled component of the composite gel becomes of lesser importance. In cases where loss of calcium occurs post-gelation, either due to slow calcium/sodium exchange in tissue, or through intentional extraction of calcium (or, as the case may be, strontium or barium) through agents such as citrate or EDTA, loss of some or all of the alginate by out-diffusion will not constitute a concern to hydrogel robustness, and may even present an advantage as alginate has potential for immunogenicity. Thus, in one embodiment alginate is extracted prior to implantation. In order to enable alginate extraction, an alginate of suitable molecular weight is used, such that it may escape from the covalently crosslinked thiol/ene hydrogel network is used. Moreover, gels formed using 1 wt. % alginate only (i.e. no TE) are either afibrotic in vivo, or dissolve within 2-4 weeks.

### Strings and Other 3-Dimensional Implants

The thiol-ene and hydrogels described herein have been found to advantageously form filament or string structures when the gelling solution is continuously co-extruded or extruded into a gelling bath. In the context of the present invention, co-extruded can be understood to refer to providing two or more separate streams that are combined at the time of extrusion, whereas an extrusion can be understood as an extrusion of already combined components, i.e. a single stream comprising a single composition. Since both techniques are suitable for the methods and processes of the present disclosure when one term is used the other is also possible, unless context specifically dictates otherwise. As used herein the term “filament” refers to an extruded hydrogel as disclosed. A filament may be referred to as a “string” and that terminology can be found herein and is interchangeable. The filament may be a composite filament, in one embodiment, a series of extrusion dies are positioned proximate to each other such that extruded strings adhere to each other as they are extruded, such that the strings form a composite filament in the shape of e.g. a flat ribbon. Further, the die may have different cross-sections, such that the filament may be e.g. a square string or oval string. In a preferred embodiment, the string has a substantially circular cross-section.

To form the filament or string, the thiol-ene polymer is extruded into a gelling bath that contains a reducing agent to induce the crosslinking of thiol-ene groups and can also include another reactive agent to induce the gelation of another hydrogel. For example, when the strings are made of alginate and thiol-ene polymers, the gelling bath would contain a reducing agent to remove the protecting group from the thiol and allow thiol-ene crosslinking to occur and a cation, preferably calcium, to gel the alginate. The solution to be gelled (precursor solution) is continuously extruded through a needle (preferably having a blunt end) directly into the gelling bath to yield the strings. The term “directly” as used herein when referring to the extrusion, means that the extrusion die (i.e., in a preferred embodiment, the needle end) from which the contents are extruded is immersed in the gelling solution or bath or sufficiently proximate to the bath that the extruded composition maintains its shape as it enters the gelling solution or bath. Suitably, the needle end can have a diameter of less than 2000 μm, preferably less than 1000 μm and more preferably less than 600 μm. The extruded hydrogel string can be considered to define a shell (i.e., outer diameter) as well as a core in which the biological material is encapsulated. The shell is denser than the core and encapsulates the contents in the core. Suitably, the core is an open network adapted to hold cells which are protected from the immune system by the polymeric shell. The strings can have an outer diameter of less than 2000, preferably less than 1000 and more preferably less than 600 μm. The size of the outer diameter is adapted to provide appropriate diffusion conditions for oxygen and chemical species to ensure that cells within the encapsulated environment can survive with sufficient access to oxygen and can secrete and receive molecules across the polymeric walls. In some embodiments, the strings have a length of a few centimetres, at least 10 cm, a few meters long, or even longer. Accordingly, an aspect ratio (length/diameter) of the strings can be at least 5, at least 10, at least 20, at least 50, at least 100, or even more. The dimensions of the strings including length and diameter can be measured using suitable microscopy techniques such as optical microscopy to then calculate the aspect ratio. Aspect ratios for these strings are defined as length divided by diameter Additional components can be included in the shell, core, or both, for example angiogenic and/or chemotactic agents. The process can be expanded to include coaxial needles (e.g. concentric) to permit formation of outer and inner gel regions of the same or different compositions, for example inner compositions more suitable for cell support and outer regions more suitable to provide mechanical strength and/or immune evasion. Accordingly, in some embodiments the shell and the core of the string have a different composition. For example, certain additives can be added in the shell and not the core, and vice versa. In addition, the polymeric composition of the shell and the core can be varied.

The thiol/ene crosslinked network is formed by combining two mutually reactive polymers (thiol containing polymer and vinyl containing) with alginate in the composite hydrogels. The generic thiol-ene reaction is presented below (Scheme 1). Additional information on thiol-ene crosslinking is described in WO2018218346 which is incorporated herein by reference in its entirety.

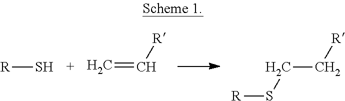

The two thiol/ene components are, for example, both based on poly(methyl vinyl ether-alt-maleic anhydride) (PMM) that were modified in one case (PMM-Spy) with a protected thiol in the form of a SPy-protected functional group (Scheme 2), and in the case of the other (PMM-VS), component, with pendant vinylsulfone (Scheme 3). In another example, the protected thiol group is PMM-cystamine (schemes 5-6).

The PMM modifications can be carried out in a polar organic solvent such as polar aprotic solvents, under conditions designed to introduce about 5 to 35 mol. %, and preferably between 10 and 30 mol. %, and most preferably between 15 and 25 mol. % of the respective thiol and vinyl groups into the anhydride-form PMM polymer. In some embodiments, the solvent is one of acetonitrile, N,N-dimethylformamide (DMF), tetrahydrofuran (THF), 1,4-dioxane, acetone, and other such solvents known to those skilled in the art. The solvent should not contain nucleophiles, for example the solvent cannot be methanol or ethanol, as these two can react with the anhydride groups of the polymer starting material. The thiol- and ene-functional PMM gel formers can be combined in different ratios, as described above, and may also be combined in different ratios of total TE polymers to sodium alginate (Scheme 4). The reaction occurs in the presence of a reducing agent to deprotect the pyridylthio groups of PMM-SPy (or other PMM-thiols) to form a free thiol (SH) group able to react. An advantage to using PMM is that it is commercially available and inexpensive, and that it is highly reactive to amines and other nucleophiles for ease of post-modifications. Alternatives to PMM include but are not limited to copolymers of itaconic anhydride or citraconic with methylvinylether, and copolymers of N-vinylpyrrolidone with maleic or itaconic or citraconic anhydride. Subsequent to functionalization, the remaining anhydride groups are hydrolyzed to render the resulting polyanionic gel formers water soluble.

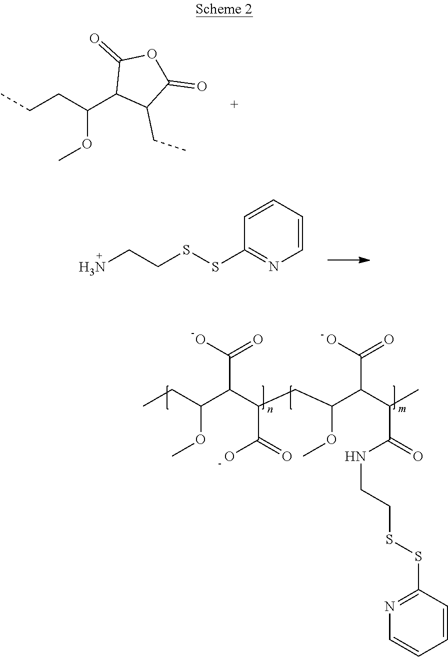

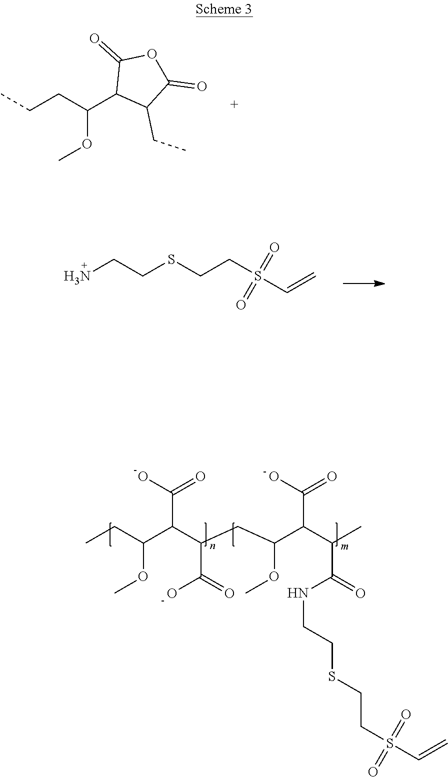

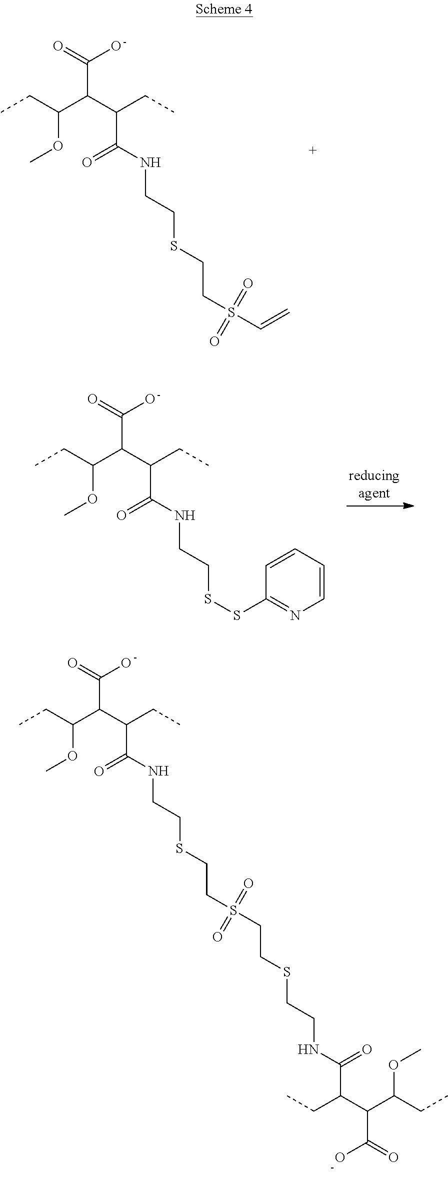

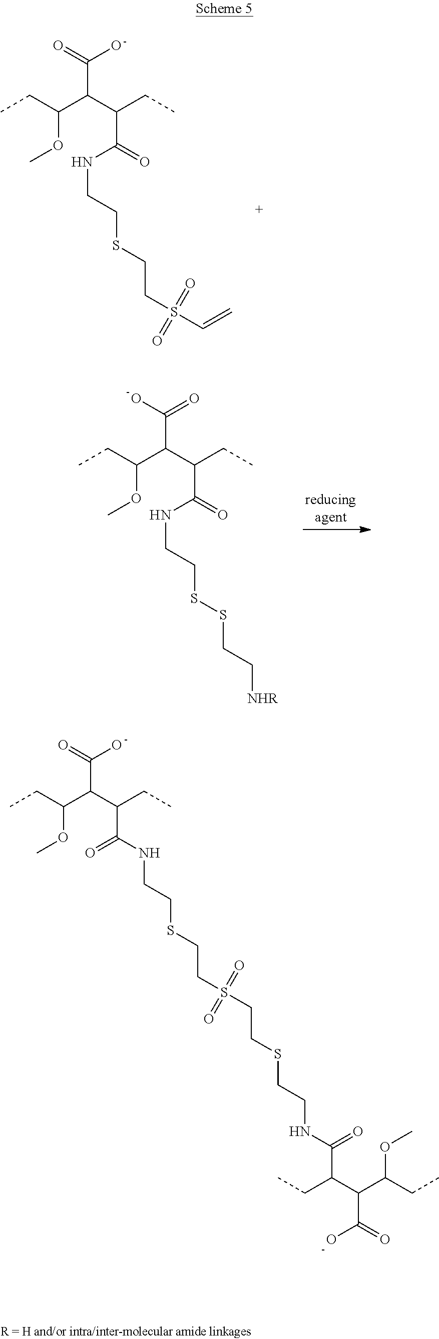

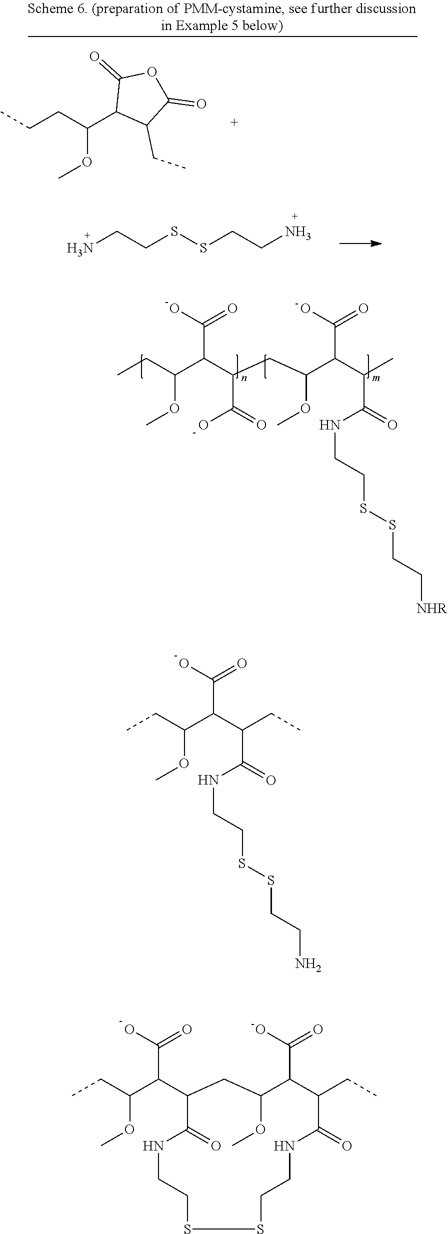

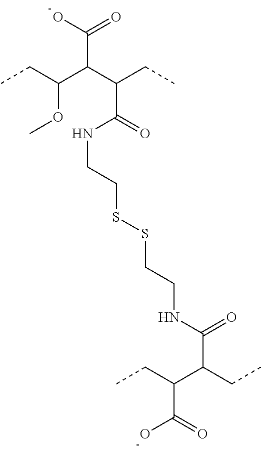

where R is H, a suitable optionally substituted alkyl group or another polymer linked by amide linkage.

As briefly explained above, the basic crosslinking chemistry of thiol (e.g. PMM-Spy) with vinyl (e.g. PMM-VS) involves a 1:1 ratio of deprotected thiol with vinyl. As previously known in the art, this 1:1 ratio of functional groups is expected to result in maximal crosslinking with minimal residual functional groups. Residual functional groups are considered undesirable as they may lead to protein binding and thus contribute to the foreign body response (FBR) of the hydrogel. The residual functional groups are also a concern with regards to toxicity towards encapsulated cells. Moreover, residual functional groups of thiol and vinyl sulfone are not maximally hydrophilic/anti-fouling, and thus do not contribute maximally towards hydration of the hydrogel.

It was surprisingly found that deviating from the 1:1 ratio of thiol and ene in favor of the ene groups can reduce the residual groups. One advantage found is that the adjustment of the ratio of thiol to ene, for example through adjustment of the wt % of comparably functionalized PMM-thiol and PMM-vinyl, allows optimization of multiple parameters/aspects of the resulting hydrogel. Specifically, using an initial excess of ene group can allow for higher conversion of a given amount of thiol groups, where the remaining excess of ene groups is subsequently capped by reaction with, e.g., cysteamine, generating hydrophilic and thus beneficial betaine groups.

The variation of the functional group ratio of protected thiol and vinylsulfone is an important tool to improve the properties of the final hydrogel with regards to optimizing the balance of mechanical robustness, hydration and swelling, anti-fouling properties, and permeability towards nutrients and oxygen while blocking cellular and molecular immune components.

The thiol to ene functional group ratio may be changed in two ways: by changing the degree of functionalization of each of the two gel-forming PMM polymers, or by adjusting the weight ratio of both polymers used, or a combination of both approaches. In practice, a simple approach is to maintain constant degree of functionalization of both thiol (e.g., PMM-Spy or PMM-cystamine) and ene (e.g., PMM-VS), and vary the wt. ratio of the polymers. A degree of functionalization of about 25 mol. % for each component allows solubilization of the two polymers in aqueous media while at the same time resulting in sufficient mechanical robustness of the crosslinked gel.

In one example, the SPy:CVS group ratio can be changed from 1:1 to 0.9:1.1, and even further to 0.7:1.3 and even 0.5:1.5, without apparent loss of final gel strength. The above example retains the total loading of CVS+SPy polymers relative to the base formulation of 1:1 ratio, though this is not a requirement—it would be acceptable to keep SPy constant and increase CVS, for example. Total loading of CVS+SPy polymers of 1.5 wt % (0.75 wt % each) has been found sufficient to form permanently crosslinked hydrogels. Mechanical strength of the gels formed increases with total CVS+SPy loading. High loadings of the gel formers (e.g. up to 3 wt. % of each) may be beneficial for encapsulation of smaller cells, including for example therapeutic or genetically modified Streptococcus thermophilus or other bacteria used for treatment of disorders of the digestive tract.

The gelation of one or more of the polymer networks forming the strings can be controlled such that multiple strings independently extruded can be linked together after extrusion by further gelation. Any three dimensional structure can be formed using this method. In one example, the gelling bath contains an amount of reducing agent that is not sufficient to induce the complete gelation of a thiol-ene polymer. For example, less than 80 mM, less than 50 mM, or from 1 to 25 mM of reducing agent such as TCEP, but less than one equivalent relative to SPy, can be included in the gelling bath, such as to provide substoichiometric reaction of SPy groups. Even string gelation with only limited levels of calcium or barium, such as 5-50 mM or preferably 10-20 mM calcium, can effect partial ionic gelation of strings and allow subsequent secondary fusion of overlapping string section through exposure to higher levels of calcium or barium, as well as exposure to TCEP or tris(hydroxypropyl) phosphine (THPP) to effect covalent crosslinking. By only partially gelling the initially formed thiol-ene polymer strings, multiple extruded string structures can be positioned to form a desired three dimensional geometry and then further gelled by adding a sufficient amount of reducing agent such as more than 1 equivalent TCEP relative to SPy, or from 1-5 equivalents, to permit deprotection of remaining amounts of SPy or other protective groups. When combined by gelation, the resulting three dimensional geometry is a single structure since the components have been covalently connected together. For example, extrusion through a set of closely spaced parallel nozzles could produce a ribbon or sheet formed of fused strings, where each individual string would retain its ability to center cell clusters. The strings can be deposited in additional shapes, including using templates, to impart desired shapes. One example may be taking the extruding string on a rolling drum to impose spiral shapes. Another example is depositing the strings into multi-layer porous sheets to further increase packing density, while still permitting diffusional metabolic exchange through channels formed by lined-up empty spaces in 2D cross-hatched patterns, and potentially enhance vascularisation. In some embodiments, the hydrogel strings are heterogeneous (denser shell than interior) to promote diffusion of oxygen and nutrients along the core of the string. In other words, the core has an open network structure that allows the diffusion of oxygen, nutrients and other biomolecules across the length of the string inside the core. In some embodiments, such a heterogeneous string structure may be enabled by asymmetric gelation of the extruded string, or else by use of a coaxial extrusion. The strings generally have a circular cross section and are extruded from circular needles, however, other shapes are contemplated herein for example a rectangular shape can be obtained with a slit and a square extrusion is also one option. In some embodiments, the strings may be heterogeneous along a length thereof e.g. cells may be introduced into the extruded stream intermittently (such as via use of a T-junction in the extrusion system). In one embodiment, the aim is to have the outer shell provide the molecular weight cutoff needed to prevent in-diffusion of cytotoxic immune molecules such as immuno-globulins, of about 150,000 Daltons, but without the whole capsule becoming dense enough to interfere with nutrient and oxygen diffusion. This concept, of having only a small outer shell cause molecular weight restriction, is standard in many technical membrane filters.

In the present disclosure when referred to a higher density, it should be noted that it is difficult to measure actual solids content of density, so instead the present disclosure provides relative densities (e.g. shell denser than interior) which can be measured by fluorescence intensity of fluorescently labelled polymer (e.g. one polymer component is labelled, or alginate can be labelled). In one example, the relative densities of the hydrogel shell versus core regions are measured by fluorescently labelling one or more of the gel forming polymers or alginate, preparing hydrogel capsules or strings or rafts, obtaining line-profiles of the intensity of the fluorescence across a cross section of the hydrogel using confocal fluorescence microscopy. The fluorescence intensity is then used as a proxy for the hydrogel density: a higher fluorescence intensity reflects a higher gel density and hence lower molecular weight cutoff. Specific molecular weight cutoffs can be measured in separate experiments where hydrogels are immersed in solutions of fluorescently labelled dextrans of specific molecular weight, and the rate and degree of in-diffusion of these dextran molecules is determined by confocal fluorescent microscopy. There are conditions, known to those skilled in the art, to create alginate hydrogels with denser shells compared to cores. These include using gelling or printing baths with increased ratios of calcium or barium chloride, to sodium chloride.

In another example, less than 1 equivalent or no reducing agent is included in the bath during extrusion. The extruded non-crosslinked or partially crosslinked mixture can then be formed into a desired shape. The crosslinking is then initiated by exposure to the reducing agent in a sufficient amount to allow gelation of the thiol and ene polymers. Optionally, gelling aids such as gelatin and other thermally gelling materials can be provided to promote the post-extrusion gelation.

The hydrogels and resulting structures described herein may be used to encapsulate biological materials such as cells, cell aggregates, cell spheroids or cell organoids. The term cell spheroids refers to an agglomeration of cells into a spheroidal shape. This differs from organoids in that organoids mimic organ function and contain different cell types and require vascularization. Mesenchymal stem cells (MSCs), including primary MSCs, immortalized MSCs, differentiated cells, and/or MSCs modified to over-express the appropriate mediators are examples of cells that can be encapsulated by the methods described herein. Other examples include but are not limited to, B-cells, pancreatic islets, liver organoids and the like. To encapsulate cells, cell aggregates or organoids, these are included in the gelling solution before extrusion and are extruded through the blunt end needle into the gelling solution. This continuous extrusion process offers flow induced centering of cell clusters such as native islets and islet reaggregates, as well as stem cell clusters, within the center of the extruded string. This keeps the encapsulated cell clusters/islets away from the wall of the gel strings, hence increasing their physical immune protection. This feature is attributed to the flow mechanics imposed by needle dimensions, solution viscosity, and cell cluster dimensions. Other approaches to ensure centering of cells and/or biologics may include co-axial extrusion of the active ingredients in the core stream with inert gel former being extruded through the annular stream. The ends of the string are made of the denser shell to fully encapsulate the core and protect any cells encapsulated in the shell from the immune systems. In some embodiments, the ends of the strings may be capped to improve the immune isolation. The strings can be implanted into a subject alone, with a support or as part of a device. The peritoneal cavity and preferably the omental pouch are suitable locations for the implants. In one embodiment, the strings are placed in the omental pouch and are sewed to form an integral part of the omental pouch. Accordingly, the strings are generally physically incorporated or entrapped into the omental pouch. In some embodiments, a nylon surgical mesh or other suitable implant supports can be used to sew the string onto a tissue such as the omental pouch. Alternatively, tissue glues and the like can be used to stabilize the strings onto a tissue (e.g. fibrinogen or thrombin glues). Use of a support may be problematic because it may increase fibrosis and decrease oxygen diffusion into and out of the strings and, accordingly, in some embodiment the strings or 3D structure formed therefrom are used without a supporting substrate. It was surprisingly found that the hydrogel strings are compatible with syringe injections. A suspension containing one or more hydrogel strings can be stored in the syringe and the strings were observed to leave the nozzle without entanglements. In one embodiment, the syringe absorbs a string from a first end and then dispenses the string from a second end (which was last to enter into the syringe). In preferred embodiments, a portion of the string in the syringe is located in the needle portion of the syringe and the reminder of the string is loosely coiled in the receptable of the syringe with a flushing solution (e.g. saline).

Benefits of the polymeric strings described herein can include but are not limited to: being easily retrievable especially for smaller cell numbers, having encapsulated islets or other cell clusters and spheroids being centered in the string, having the capacity to be pre- or post-modified with desirable attachment functions or other biomolecules, and having a long-term integrity due to the crosslinked TE network that allows multiple media exchanges without weakening of the hydrogel.

String extrusion can take place into a full strength gelling bath with 100 mM calcium chloride which causes instantaneous formation of a calcium alginate skin on the emerging string, preventing string segments from adhering to each other. This mimics the process that prevents bead adhesion in the gelling bath and facilitates forming long sections of smooth string.

Conversely, decreasing the calcium chloride concentration in the gelling bath to about 25 mM calcium chloride allows string segments formed within seconds of each other to adhere to each other, thus enabling partial fusion of overlapping sections of string. This facilitates 3D bioprinting of, e.g., cross-hatched patches of string—a pattern useful for compact assembly of strings containing cell clusters. Such patches still enable metabolic exchange by passive diffusion and vascularization through the channels within the patch. To achieve such embodiments, the calcium chloride level in the gelling bath can be reduced from the standard 100 mM to between 10 and 50 mM. Such crosshatched patches may extend into three dimensions, e.g., through overprinting of two or more cross-hatched patterns on top of each other, provided the open spaces line up sufficiently to become through-channels for metabolic exchange by passive diffusion and, eventually, vascularization. Similarly, the TCEP level in the gelling bath can be reduced to a point where gelation is retarded enough to enable partial fusion of overlapping strings. An example is reducing the TCEP level to zero during string or patch formation and initiating thiol/ene crosslinking by post-exposure of the sheet to TCEP in a separate step. This may include incorporating other gelling aids such as gelatin and other thermally gelling materials.

In some embodiments, the hydrogel string is heterogeneous, e.g., has higher network density at the string surface. This can be achieved by coating or re-enforcing the external surface of the hydrogel string post-gelation for example with polycation, further crosslinking, introducing hydrophobicity. Other methods may include heterogeneous gelation, such as through use of a sodium-free calcium gelling bath, or indeed through use of coaxial extrusion. This may increase surface strength, and favour lateral (along string center) diffusion of oxygen and nutrients and possibly cells. In one embodiment, this would allow encapsulation of individual cells or small cell clusters, followed by their expansion and self-aggregation into a “cell string” along the center of the hydrogel string, potentially optimizing cell loading and metabolic connection. When the strings are heterogeneous (denser shell than string core), this allows maximization of strength versus gel loading.

### EXAMPLES

### Example 1: Thiol-Ene Crosslink Network Synthesis

The thiol/ene crosslinked network was formed by combining two mutually reactive polymers with alginate in the composite hydrogels. The two thiol/ene components are both based on 80 kDa poly(methyl vinyl ether-alt-maleic anhydride) (PMM) that were modified in one case (PMM-SPy) with a SPy-protected thiol, and in the case of the other component (PMM-VS), with pendant vinylsulfone.

These PMM modifications were carried out in acetonitrile solution, under conditions designed to introduce about 5 to 35 mol. %, and preferably between 10 and 30 mol. %, and most preferably between 15 and 25 mol. % of the respective Spy and Ene groups into the anhydride-form PMM polymer. Briefly, PMM in the anhydride form (1 g, 6.41 mmol anhydride) was dissolved in 20 mL acetonitrile and transferred to a round bottom flask equipped with a magnetic stir bar. To the vigorously stirring reaction mixture, triethylamine (TEA, 360 uL), followed by either S-(2)-pyridylthio cysteamine hydrochloride (0.3 g; 1.35 mmol) or cysteamine vinyl sulfone hydrochloride (0.36 g; 1.55 mmol) was added drop-wise to prepare PMM-SPy or PMM-VS, respectively. After overnight reaction, the reaction mixtures were transferred into 6-8 kDa molecular weight cut-off tubing and placed in dialysis water baths containing 1 wt. % sodium chloride in distilled water for a total of 2 changes per day for 2 days, followed by 2 distilled water bath changes per day for 2 days. UV-vis absorbance measurements of the dialysate were conducted to confirm no small molecule impurities were detected. The dialyzed polymer solutions were freeze dried, resulting in PMM-SPy and PMM-VC as a light pink solids. ¹H NMR in D₂O was conducted to confirm modification percentages at approximately 20 mol %.

To prepare TEC 0.9:1.1 (TEC 911) capsules, PMM-Spy and PMM-VS were combined in a weight ratio of 0.9:1.1 respectively while maintaining the overall polymer loading (i.e. 2% total polymer loading). Capsules were first prepared to test the chemistry before moving to the formation of strings. PMM-SPy (36.0 mg) and PMM-VS (44.0 mg) were dissolved in 1.80 mL of 35 mM N-2-hydroxyethylpiperazine-N-2-ethane sulfonic acid (HEPES) buffered saline (HBS) and adjusted to pH 7.6 with 1 M sodium hydroxide. Following pH adjustment, the polymer solution was increased to a total volume of 2.00 mL by adding 35 mM HEPES buffered saline. The polymer solution was mixed with 2.00 ml of 2 wt. % sodium alginate (Novamatrix PRONOVA™ UP MVG) for a total of 5 min to allow for adequate mixing. The mixture was subsequently filtered through a 0.22 μm sterile filter and then transferred into a 3 ml syringe, which was fitted with a 20 G, 14 G outer coaxial needle (Ramé-Hart Instrument Co.) and positioned into a vertically oriented syringe pump (Harvard Apparatus Pump 11 Elite) inside a biosafety cabinet. Capsules were prepared by extruding through the inner needle the polymer solution into 50 mL of constantly stirred sterile gelling bath solution containing 100 mM calcium chloride, 35 mM HEPES buffer, 0.45 wt. % sodium chloride, and 3.5 mM tris(carboxyethyl) phosphine hydrochloride (TCEP) adjusted to pH 7.6. Liquid extrusion rate through the inner needle was set to 15 mL/H and air flow through the outer needle was set to 2.5 L/min and the tip of the inner needle was wiped every 1.5 minutes to remove any dried alginate. The capsules were treated by constant swirling for 10 minutes in a 30 mM L-cysteine and 3.5 mM TCEP solution adjusted to pH 7.6, followed by 2 washes with saline. The capsules were left in the gelling bath for a total of 15 min after extrusion, followed by 2 washes with saline. The capsules were suspended in a 1:1 v/v capsules:saline ratio for implantation.

The additional PMM-VS allowed increased conversion of the thiol groups liberated from the PMM-Spy. This also led to the presence of an excess of residual PMM-VS functional groups which can then be beneficially reacted with post-modification agents such as cysteine to introduce a correspondingly larger proportion of anti-fouling functionality. Other relevant ratios identified were 0.8:1.2, and 0.7:1.3, and 0.5:1.5. These maintained constant overall polymer loading and hence gel former viscosity (which is desirable from the perspective of microfluidic flow properties, cell shear stress, and 3D printing).

### Example 2: TEC 0.9:1.1 Implants into Healthy Mice

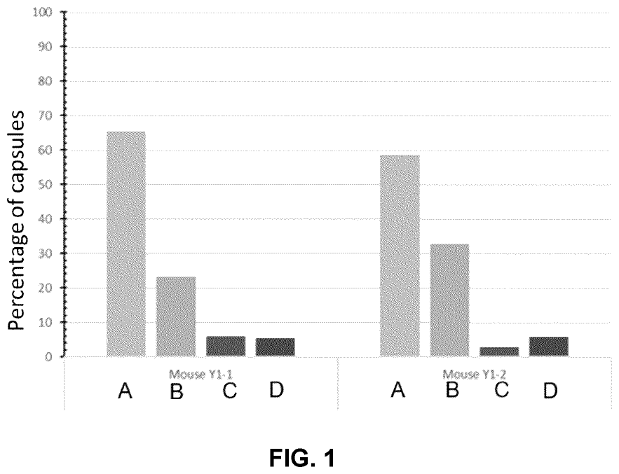

This example provides evidence of anti-fouling functionality of implants according to an embodiment of the present invention. PMM-Spy and PMM-VS were combined in a ratio of 0.9:1.1 to form blank capsules, which were treated with 30 mM cysteine and implanted into intraperitoneal space (I.P.) of healthy C57BL/6 mice. Two weeks later, the capsules were explanted with 50-80% recovery rate, and found to have minimal fibrosis. The detailed fibrosis results are summarized in FIG. 1.

### Example 3: Encapsulation of Islets and Cell Function and Immunoprotection In Vivo for 0.9:1.1 TEC

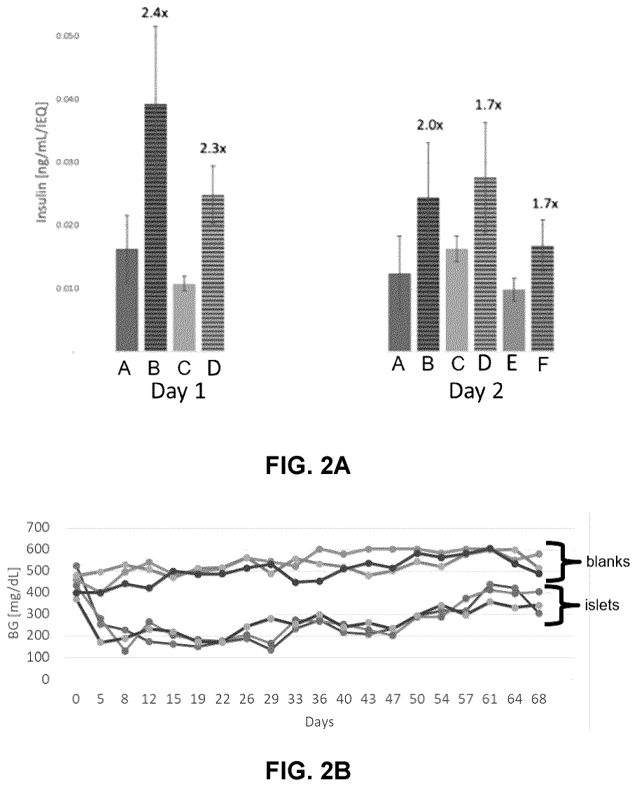

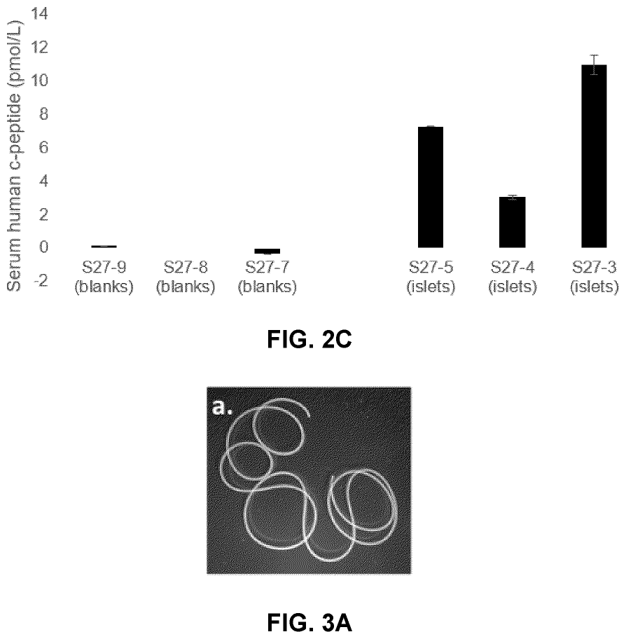

This example provides evidence of cell functionality in vitro and in vivo when encapsulated in a 0.9:1.1 thiol:ene hydrogel. This formulation was found to have improved insulin release by glucose-stimulated insulin secretion (GSIS) compared to a 1:1 thiol:ene obtained hydrogel or an only alginate hydrogel. Glucose stimulation triggered a two times improved insulin release which indicates a quick mediator exchange (FIG. 2A), and in vivo, it enabled human islet functionality up to 50 days (which is longer than reported in literature for free non-encapsulated islets (e.g. Qi, M., Mørch, Y., Lacik, I., Formo, K., Marchese, E., Wang, Y., . . . & Strand, B. L. (2012). Survival of human islets in microbeads containing high guluronic acid alginate crosslinked with Ca²⁺ and Ba²⁺. Xenotransplantation, 19(6), 355-364. and Qi, M., Strand, B. L., Mørch, Y., Lacik, I., Wang, Y., Salehi, P., . . . & Oberholzer, J. (2008). Encapsulation of human islets in novel inhomogeneous alginate-ca²⁺/ba²⁺ microbeads: in vitro and in vivo function. Artificial cells, blood substitutes, and biotechnology, 36(5), 403-420), namely up to 8 times longer), as indicated by rapid return of high blood glucose of diabetic immunocompetent mice to normoglycemia, sustained for ˜50 days (FIG. 2B) and increased level of human C-peptide in the serum of immunocompetent diabetic mice that received human islets encapsulated with the 0.9:1.1 of thiol:ene hydrogel formulation treated with cysteine (FIG. 2C). The cells remained functional and reduced the blood glucose to normal levels (200 mg/dL+/−SD of 50 mg/dL) for up to 68 days (FIG. 2B).

### Example 4: TEC Strings

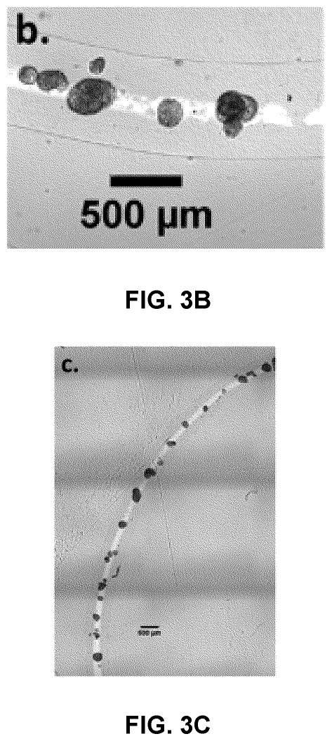

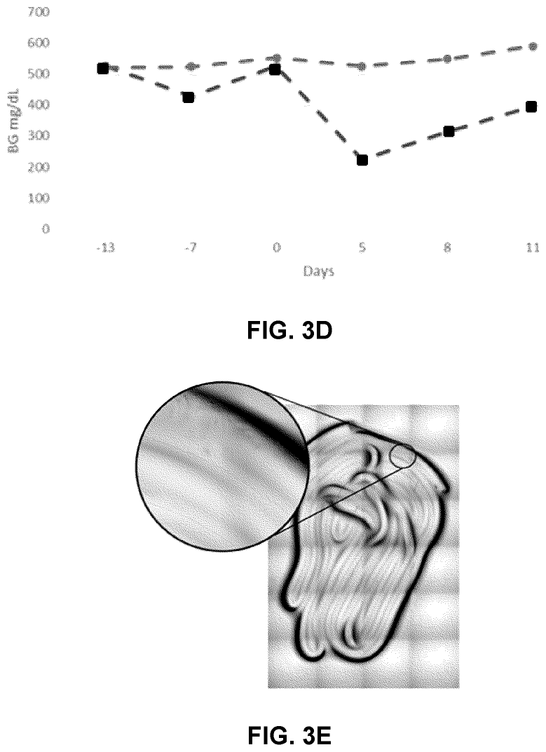

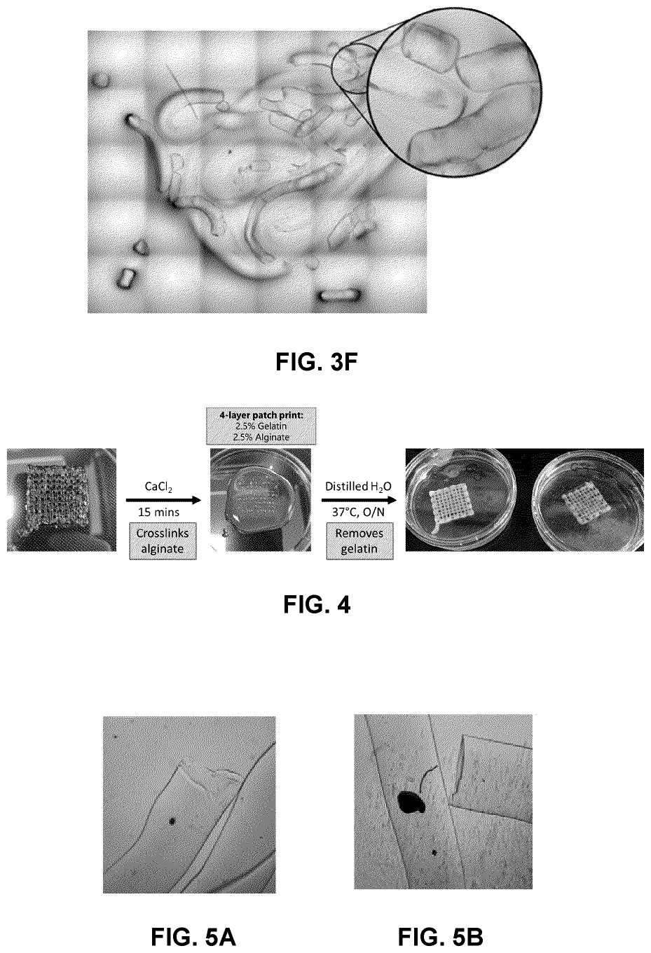

The formulations (0.9:1.1, 0.8:1.2 and 0.7:1.3 ratios of thiol:ene) were extruded in a continuous fashion into gelling baths to form strings. The strings are an advantage for retrievable implantable devices, which may permit better prevention of cell-cell contact due to centering of cell clusters through microfluidic effects, and have other processing advantages such as avoiding the need for constant sterile airflow. Examples include gel former solutions containing 0.5-4 wt. % sodium alginate as well as 0.5-6 wt. % each of thiol and ene polymers. The formation of continuous gel strings greater than 1 meter in length, and 0.2 to 1.5 mm in diameter was demonstrated. A cysteine treatment (first wash in saline then exposing the string to 10-60 mM cysteine in HBS) was performed to convert any remaining vinylsulfone groups into a non-reactive group thereby obtaining a thiol-ene cysteine (TEC) polymer. An image of a cysteine-treated fluorescent 0.55 mm diameter 1.2% alginate, 1.5% 0.7:1.3 TEC string greater than 1 m in length is shown in FIG. 3A. It was further shown that these strings could incorporate rat islets that remained viable in vitro for multiple days. Significantly, it was noted that these rat islets consistently appeared to be centered within the extruded strings (FIG. 3B-3C). TEC strings containing rat islets were implanted by injection into the peritoneal cavities of diabetic mice, and observed short-term reduction of blood sugar levels (up to 11 days), compared to mice receiving blank TEC strings (FIG. 3D). Finally, it was shown that after the TEC strings were implanted in the peritoneal cavities of immuno-competent mice and were explanted quantitatively after two weeks, the explanted strings showed little cell attachment along the length of the strings. Some breakage of strings was observed, with broken ends accumulating more significant fibrosis, attributed to roughness of the broken edges (FIGS. 3E-3F). Strings are easy to implant into I.P. space. Such breakage can be mitigated by incorporation of additives such as hyaluronic acid that increase lubricity without reducing strength. Moreover, implantation into an omental pouch in larger animals and indeed humans would include abdominal insufflation which would decrease the risk of string damage during injection.

While strings are prototypes of retrievable devices, the length of string required for transplantation of a therapeutic dose of islets is approximately 15 cm for a mouse (1000 IEQ/25 g mouse), and possibly up to 40 m or longer for a human (about 300,000 IEQ/75 kg human). As a result, the string extrusion was adapted to form patches that combine high surface area (required for survival and good metabolic connection of the islets) with condensed form factor. This approach resulted in hand-sized patches comprising a human therapeutic dose of islets. The microporosity of the patches were tuned to maximize vascularization and integration with host tissues. Advantages of the present devices include the TE and TEC compositions, and the islet centering phenomenon in the strings.

An advantageous feature in string formation is the ability to microfluidically center the islets and to ensure strong fusion between strands of overlapping string extruded after 3D bioprinting into a 3D patch device. This is challenging to achieve because the traditional chemistry is designed to cause immediate gelling by calcium as the string enters the gelling bath, such that overlap of new string with string extruded even a few seconds earlier is compromised by the formation of a gelled skin on the outer surface of the earlier string section. The inventors have developed methods to overcome this. One method is to print the scaffold directly into a negative mould of the patch shape that contains the gelling bath solution, which would inhibit the string moving around into undesired positions during the printing process. Another method is to print the strings into a viscous support bath (e.g., FRESH™ (freeform reversible embedding of suspended hydrogels) material or equivalent) that can support and maintain the shape of the printed material until solutions can be added to induce crosslinking of the alginate or TEC polymers (using CaCl₂) or a reducing agent, TCEP, respectively). A further method is to print a supporting biomaterial along with or even instead of alginate in the TEC/alginate formulations. Such a material, examples of which include gelatin, Pluronic F127, etc., can allow for bioprinting onto a wide variety of surfaces without use of a supporting bath, with the material itself supporting the shape of the printed material until crosslinking of the alginate or TEC can be initiated via the addition of CaCl₂) or TCEP, respectively. Importantly, these materials can replace alginate entirely in the formulation, if desired, and can be removed post-crosslinking. For example, specific concentrations of gelatin or Pluronic F127 can be removed via heating the patch to 37° C. or by cooling to 4° C., respectively. Proof of concept of this last strategy is shown in FIG. 4. FIG. 4 shows a bioprinted 4-layer cross-hatched patch comprised of alginate and gelatin that was printed using an inexpensive modified commercial 3D printer onto a petri dish. The gelatin here supports the 3D structure in a dry state throughout the print and after the print was finished, it was soaked in a CaCl₂) solution for 15 minutes to crosslink the alginate and then the patch was removed and placed in a solution of distilled water or saline at elevated temperature (37° C.) to remove the majority of the gelatin, leaving an alginate patch. Analogous methods could be used using gelation, Pluronic F127, etc., with TEC alone (its crosslinking mechanism similarly operates within seconds), TEC/alginate, or TEC with other additives to generate implantable patches. A similar methodology can be used to obtain similar 100% TEC patch formats. In addition, the overlapping string sections as extruded can be held in place by having present in the primary extrusion bath a reduced concentration of calcium, such as 1-50 or preferably 2-20 mM, enough to retain the extruded string segments but still low enough to enable fusion of overlapping string segments. Exposure to THPC once the patch extrusion is complete would trigger covalent crosslinking to lock the string segments in place permanently. Finally, a substoichiometric amount of THPC reducing agent may be added to the primary gelling bath to initiate partial gelation upon extrusion, but with reservation of the majority of the crosslinking functionality for post-gelation after extrusion is complete.

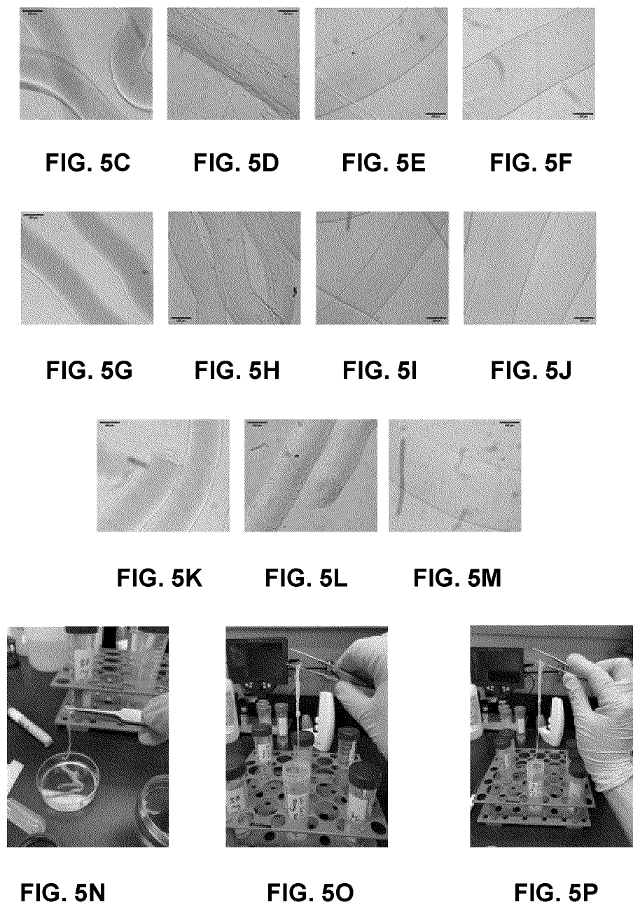

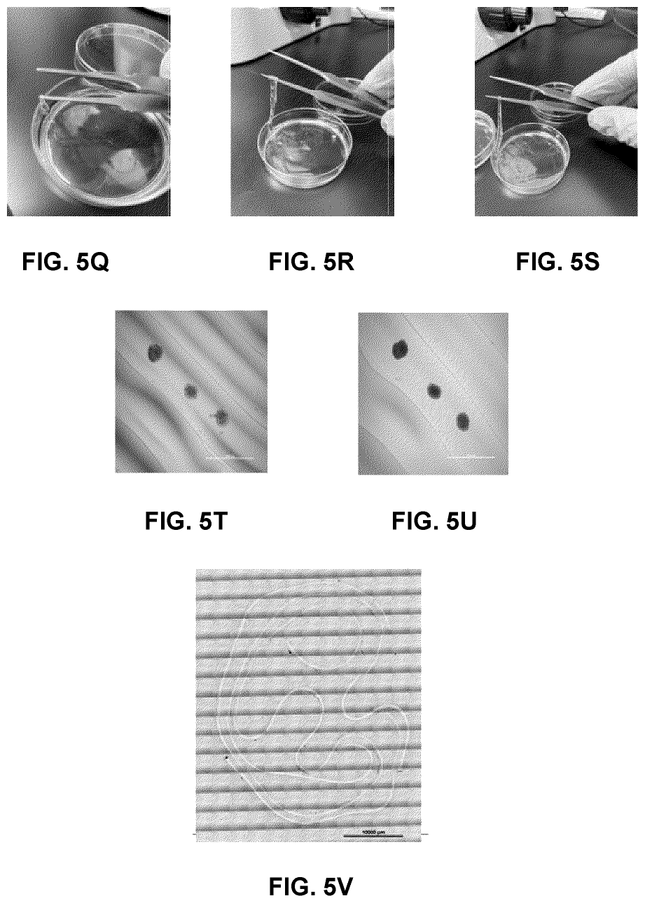

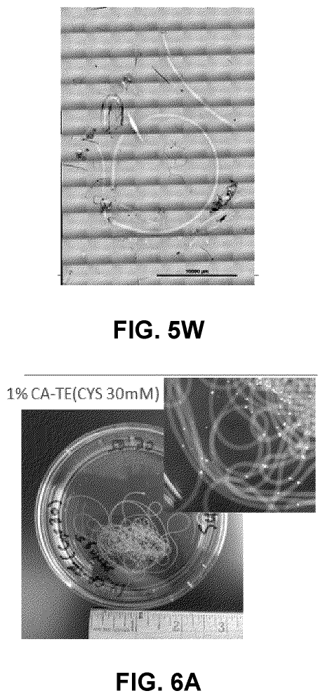

String formation was performed by extrusion with a syringe pump, the syringe preferably having a blunt end. Islets were encapsulated using TEC strings at 1% or 2% CA-TE (with cysteine 30 or 60 mM, labeled as CA-Tex/CysY where x=the percentage of CA-TE and Y=the cysteine mM), at 1 or 2 islet/inch. FIGS. 5A-5B show sharp edges of “cut” string ends demonstrated radial in-diffusion of calcium leads to radial density gradient of both calcium alginate and TE networks (CA-TE1.0/Cys30 was used). Unless otherwise specified, when a percentage associated to CA-TE is specified, it refers to the TE concentration with CA being fixed at 1%. Good strength combined with good permeability across and within the string shell was obtained. String swelling for 0.75%, 1.0% and 1.5% TE was observed after citrate challenge for up to 45 min (FIGS. 5C-5M). As shown in Table 1, swelling of 340-450% was observed. The mechanical robustness before and after citrate exposure were compared for strings of 0.75%, 1.0% and 1.5% TE (FIGS. 5N-5S). Before citrate exposure, at 0.75% TE the string broke at 4 cm when lifted with a tweezer, and at 1.0% and 1.5% TE the entire length was supported by the tweezer. After citrate exposure, the string broke at 1 cm, 3 cm, and 5 cm for 0.75%, 1%, and 1.5% TE respectively. The TEC strings were shown to be mechanically robust and maintained structure after stripping calcium from alginate (FIGS. 5T-5U). An example of a string encapsulating islets is shown in FIGS. 5V-5W. A 30 cm CA-TE_{1%} (CYS_{30 mM} strings with no cells were injected intraperitoneally in healthy mice and retrieved after 7 days.

TABLE 1
TE swelling measurements

| Measure | 0.75% TE | 1.0% TE | 1.5% TE |
|---|---|---|---|
| Diameter | 852 +/− 79 | 763 +/− 23 | 867 +/− 97 |
| Before | | | |
| Citrate | | | |
| (μm +/− SD) | | | |
| Diameter | 1453 +/− 128 | 1243 +/− 150 | 1332 +/− 140 |
| After 45 min | | | |
| (μm +/− SD) | | | |
| Diameter | 1.71 +/− 0.13 | 1.63 +/− 0.12 | 1.54 +/− 0.15 |
| Ratio | | | |
| Volume % | 500% | 433% | 340% |
| increase | | | |

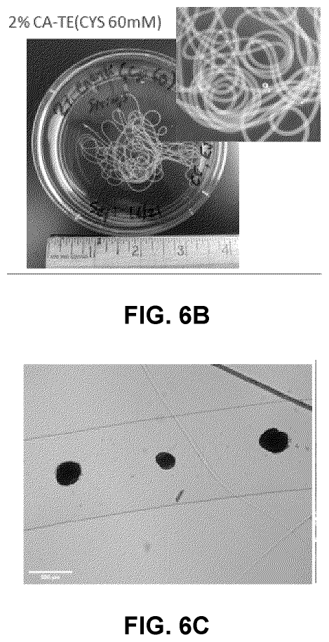

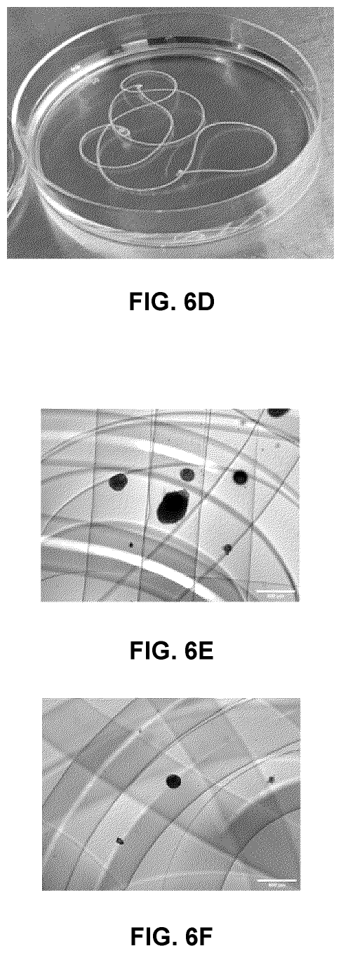

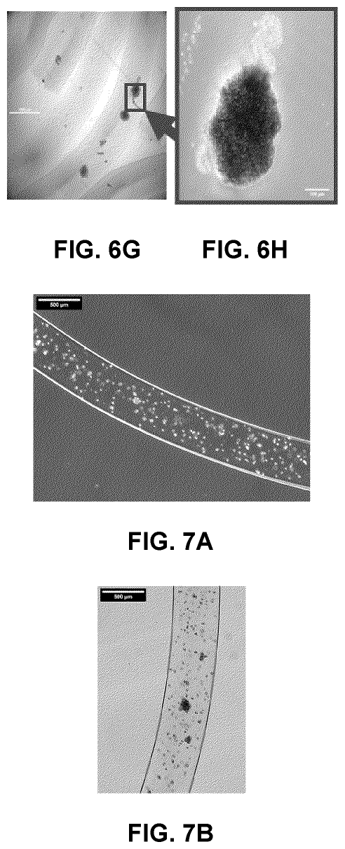

The appearance of the strings is shown in FIGS. 6A-6E. For CA-TE1.0/Cys30 two islets per inch were obtained and for CA-TE2.0/Cys60 one islet per inch was obtained (FIGS. 6A-6D). Islets were centered and viable in vitro for up to 1 week, in diameter ranges of ˜700 μm for CA-TE1.0/Cys30 (FIGS. 6E-6F). Viability of cells was indicated by green fluorescence of live/dead staining (FIGS. 6G-6H). Rat islets 4 days after encapsulation were stained by Ethidium/Calcein AM live/dead stain. In addition, these were implanted intraperitoneally in STZ-diabetic mice. Blood glucose control was observed for one week (Table 2). Within 5 days, blood glucose dropped, albeit a temporary effect that resides within one week. The strength of the strings in the intraperitoneal space of healthy mice was also tested. After 2 weeks, strings explanted from healthy mice implanted intraperitoneally 2 weeks earlier were well-retrieved but appeared shorter with broken ends. This is attributed to injection into small I.P. spaces in mice without benefit of insufflation, which would mitigate this concern in larger animals and indeed, humans.

TABLE 2
Blood glucose levels in mice having received a string transplant

| | | | | control | control | control |
|---|---|---|---|---|---|---|
| | string | string | string | string | string | string |
| | with | with | with | without | without | without |
| day | islets | islets | islets | islets | islets | islets |
| −9 | 477 | 495 | 486 | 369 | 491 | 430 |
| −2 | 407 | 547 | 477 | 428 | 389 | 409 |
| 0 | 358 | 400 | 379 | 360 | 419 | 390 |
| 5 | 259 | 252 | 256* | 297 | 486 | 392 |
| 8 | 340.2 | 369 | 354.6 | 307 | 401.4 | 354.2 |
| 12 | 342 | 328 | 335 | 340 | 437 | 412 |

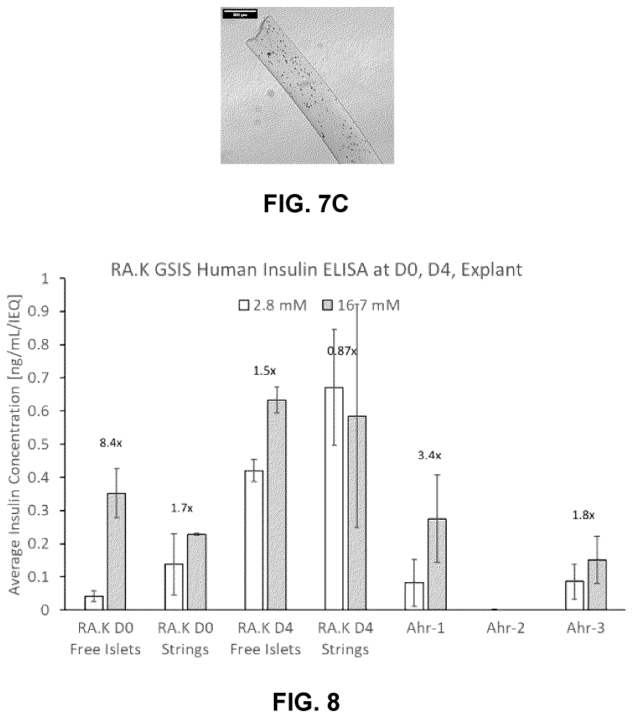

Strings made of 1% TEC Cys 30 mM composition, diameter 566 μm (SD 6 μm), were used to encapsulate stem cells cluster of diameter 54 μm (SD 15 μm). The characteristics of the strings used are summarized in Table 3. As strings were extruded, it was noted that cell clusters, but not single cells, were well-centered (FIGS. 7A-7C).

TABLE 3
Characteristics of the strings
String Stats

| String Diameter (n = 20) | 566 μm (SD 6 μm) | |
|---|---|---|
| Cluster Diameter (n = 70) | 54 μm (SD 15 μm) | |
| Cell Loading | ~4k clusters/mL | ~20k clusters/mL |

| | |
|---|---|
| Total available | ~12.5k clusters (1 well) |

SD = standard deviation

### Example 5: Synthesis of PMM-Cystamine

In another example, PMM-cystamine (PMM-Cy) was used as a precursor to the reactive thiol polymer instead of PMM-SPy. To prepare PMM-cystamine targeting 10 mol % degree of functionalization, PMM in the anhydride form (1 g, 6.41 mmol anhydride) was dissolved in 75 mL of N,N-dimethylformamide (DMF) and transferred to a round bottom flask equipped with a magnetic stir bar. To the vigorously stirring reaction mixture, triethylamine (TEA, 360 uL) was added, followed by a solution of cystamine dihydrochloride (0.144 g, 0.64 mmol) in 4 mL of a 1:1 v/v of DMSO:DMF in a drop-wise manner. After overnight reaction, the reaction mixtures were transferred into 12-14 kDa molecular weight cut-off tubing and placed in dialysis water baths containing 2 wt. % sodium chloride in distilled water for a total of 2 changes per day for 2 days, followed by 2 distilled water bath changes per day for 1 day. UV-vis absorbance measurements of the dialysate were conducted to confirm no small molecule impurities were detected. The dialyzed polymer solutions were freeze dried, resulting in PMM-cystamine as a light pink solid. ¹H nuclear magnetic resonance (NMR) in D₂O was conducted to confirm purity of the polymer with no small molecule impurities detected. Elemental analysis of the sample was conducted, resulting in 1.5% N, 38.28% C, 5.03% H, and 3.63% S which calculated to about 13% modification of cystamine units of the PMM backbone. It should be noted that the reaction conditions, in particular the concentration of the reaction, played a key role in forming PMM-cystamine as a soluble product. Under concentrated conditions (i.e. concentrations greater than 150 mM of PMMAn), cystamine reacts with PMMAn with a higher probability of forming the inter-polymer chain amide linkages which effectively forms cross-linkages (Scheme 6 second line compound on the right), resulting in an insoluble product. By diluting the reaction mixture (i.e. diluting by from 2.5× to 4×dilution factor of the concentrated concentration previously mentioned), cystamine has a higher probability to react with intra-polymer chain anhydride groups, resulting in macro-cyclic amide linkages which results in a soluble product (Scheme 6 second line compound in the middle). The structure shown in the compound in the middle of the second line in scheme 6, is an example of a reaction with a neighbouring anhydride group, however larger macrocycles are also possible with reaction of anhydride groups on the same polymer chain that are further away. There is potential for cystamine to mono-substitute and react with a single anhydride, resulting in a pendant amine (Scheme 6 second line compound on the left), however this result is less likely as the reaction is left overnight and there is an excess of anhydride groups available for reaction. The reaction yielding the macrocycles is preferred, as this results in a soluble product, and also two reactive polymeric thiols per mol of cystamine upon reduction that are available for forming thiol-ene linkages in subsequent gelation reactions. The reaction conditions described were found to be optimal in forming soluble PMM-cystamine as a gel former.

### Example 6: Preparation of TEC-Cy Gel Forming Solutions to Prepare Hydrogel Capsules, Strings, and Patches

To prepare TEC-cy hydrogel capsules, PMM-cystamine (10 mol % degree of functionalization) and PMM-VS (20 mol % degree of functionalization) were combined at a 1:1 mol ratio of thiol to vinyl groups while targeting a total wt./v % of preferably 1.5%, along with a sodium alginate at a final concentration of 1%. For example, to prepare 4 mL of the TEC-cy gel forming solution, PMM-cystamine (28.3 mg) and PMM-VS (31.7 mg) were dissolved in 2 mL of HBS, with pH adjusted to pH 7.4 with 1 M NaOH. To this solution, 2 mL of 2% sodium alginate was added and vortexed to mix to form a final concentration of 1.5% thiolene polymers and 1% alginate. The gel forming mixture was filtered using a 0.22 μm syringe filter prior to formation of hydrogel capsules, strings, and patches.

### Formation of TEC-cy Hydrogel Capsules

To prepare TEC-cy hydrogel capsules, the TEC-cy gel forming solution as described above was loaded into a syringe and fitted with a 20 G, 14 G outer coaxial needle (Rame-Hart Instrument Co.) and positioned into a vertically oriented syringe pump (Harvard Apparatus Pump 11 Elite) inside a biosafety cabinet. Capsules were prepared by extruding the polymer solution through the inner needle into a gelling bath solution containing 100 mM calcium chloride, 35 mM HEPES buffer, 0.45 wt. % sodium chloride. Liquid extrusion rate through the inner needle was set to 15 mL/h and air flow through the outer needle was set to 2.7 L/min and the tip of the inner needle was wiped every 1.5 minutes to remove any dried alginate. Following completion of the extrusion, the capsules were cured for 15 min in the gelling bath. The capsules were transferred to a solution of TCEP to cleave the disulfide groups and trigger covalent crosslinking through thiol-ene reaction with the vinyl sulfone groups. The capsules were treated by constant swirling for 10 minutes in a 25 mM L-cysteine solution adjusted to pH 7.6, followed by 2 washes with saline. The capsules were left in the gelling bath for a total of 15 min after extrusion, followed by 2 washes with saline. The capsules were suspended in a 1:1 v/v capsules:saline ratio for implantation.

### Formation of TEC-cy Hydrogel Strings

To prepare the TEC-cy hydrogel strings, the TEC-cy gel forming solution was loaded into a syringe and fitted with a 20G blunt tip needle. The syringe was fitted into a vertically oriented syringe pump and the needle tip was submerged into a gelling bath containing a solution of 100 mM calcium chloride, 35 mM HEPES, and 0.45 wt. % sodium chloride at pH 7.4 or a barium gelling bath with 10 mM barium chloride, 35 mM HEPES, and 0.45 wt. % sodium chloride at pH 7.4. The polymer solution was extruded into the gelling bath at a liquid flow rate of 10 mL/h.

### Example 7: Encapsulation of Islets

### Encapsulation of Islets in TEC-cy Capsules

To prepare islet-containing capsules, 2 wt. % sodium alginate in HBS was first diluted to a concentration of 1.5 wt. % by addition of HBS followed by 5 min mixing with a transfer pipette. The resulting alginate solution was sterile filtered (0.22 μm filter), then further diluted with a suspension of islets in HBS to form a final concentration 1 wt. % sodium alginate containing islets. The alginate-islet mixture was gently mixed with a transfer pipet for 5 min. The suspension was transferred into a 1 ml syringe, which was then loaded into a syringe pump set at a liquid flow rate of 15 mL per hour. The syringe was fitted with a 20G/14G coaxial flow blunt tip needle (Rame-Hart) and the side arm was connected to an air line with an air flow rate adjusted to 2.5 L/min. The islet containing sodium alginate solution was extruded into a stirred gelling bath solution containing 100 mM calcium chloride, 77 mM sodium chloride, and 35 mM HEPES at pH 7.4. Following extrusion, the beads were collected into 50 mL centrifuge tubes and they were allowed to cure for 15 min on ice, with occasional mixing. After incubation, the beads were allowed to settle to remove the supernatant. The beads were washed twice with saline at a 3:1 bead to solution ratio prior to coating procedures as described in the following sections.

Implantation into C57BI/6J Mice

C57BI/6J mice were weighed and anesthetized using isofluorane. Once fully anesthetized, the animal was shaved along the abdomen and cleaned with alcohol wipe in preparation for intraperitoneal injections. Under anesthesia, the animal was injected subcutaneously with Buprenorphine (0.05 mg/kg) for pain and intradermally with Bupivacaine (8 mg/kg) for local anesthesia at site of injection. The animal then received dose of either encapsulated islets (800 IEQ) or blank capsules in 0.9% NaCl (saline) injected intraperitoneally with a 16G needle (total injection volume of 1 mL). The syringe was loaded with 1-2 mL of saline post injection and ejected into a clean 50 mL tube to collect any remaining capsules. The animal was allowed to recover on a heating pad and observed for any discomfort.

At experimental endpoint, the animals were humanely euthanized, and blood collected via cardiac puncture. The capsules were explanted washing the peritoneum with 0.9% NaCl (saline) into a stainless-steel kidney collection dish. Using a plastic Pasteur transfer pipette, capsules were collected into a fresh 50 ml tube. Capsules were washed twice with fresh 0.9% NaCl (saline) to remove any debris or unattached cells. Capsules were then transferred to 4% v/v formalin (methanol-free) to reach a 10:1 formalin solution to capsule ratio. Capsules were then imaged for pericapsular fibrotic overgrowth (PCO) analysis as indicated below.

To measure islet functionality by insulin secretion, low (2.8 mM) and high (16.7 mM) D-glucose Krebs-Ringer Bicarbonate HEPES (KRBH) buffer at pH 7.4 (135 mM NaCl, 3.6 mM KCl, 5 mM NaHCO₃, 0.5 mM NaH₂PO₄·2H₂O, 0.5 mM MgCl₂·6H₂O, 1.5 mM CaCl₂·2H₂O, 10 mM HEPES, and 0.1% BSA) was made and sterile filtered using 0.22 μm filter before use.

For each round of islets and encapsulations, islets were stimulated in vitro in a 24-well sterile plate. Briefly, 10 free islets or 10 capsules with islets at ˜100 μm diameter were distributed in 24-well plate (10 islets/well) in triplicate in 1 mL of warm supplemented RPMI 1640 media. Using a digital benchtop microscope, 10x˜100 μm diameter free islets were handpicked into prepared wells using a p100 or p200 micropipette. Similarly, 10 capsules with similar sized islets were added to prepared wells in triplicate. Once all the samples were added to the appropriate wells, the media was replaced with 1 mL of warmed 2.8 mM glucose KRBH buffer. Images of free and encapsulated islets were taken using a conventional bright field microscopy for IEQ calculation as described above. The 24-well plate was incubated at 37° C., 5% CO₂ humidified incubator for 1 hour. The 2.8 mM glucose KRBH buffer was replaced with fresh 1 mL 2.8 mM glucose KRBH buffer and incubated as before for 1 hour. The 2.8 mM glucose KRBH buffer was collected into labelled 1.5 mL microfuge tubes as baseline islet insulin secretion measurements. 1 mL of warmed 16.7 mM glucose KRBH buffer was added on the free and encapsulated islets, and incubated as before for 1 hour. The 16.7 mM glucose KRBH buffer for islet insulin secretion after stimulation. Samples were stored at −20° C. until further use.

Insulin secretion was measured via the Ultra-sensitive Rat Insulin enzyme linked immunosorbent assay (ELISA) kit from Crystal Chem (Downers Grove, IL, USA) and normalized to the IEQ for each well.

### Results

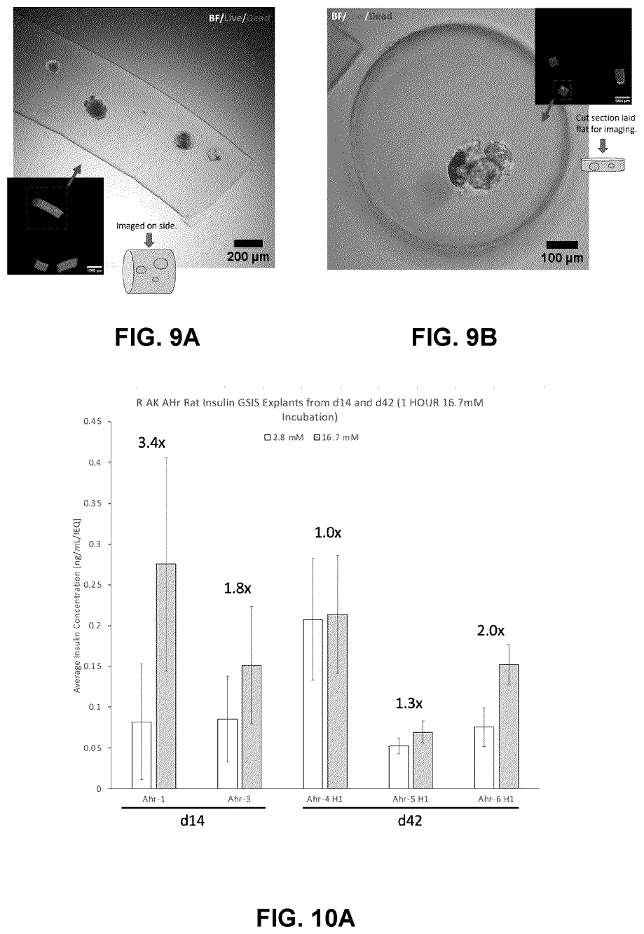

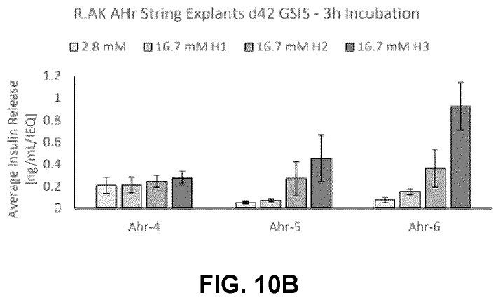

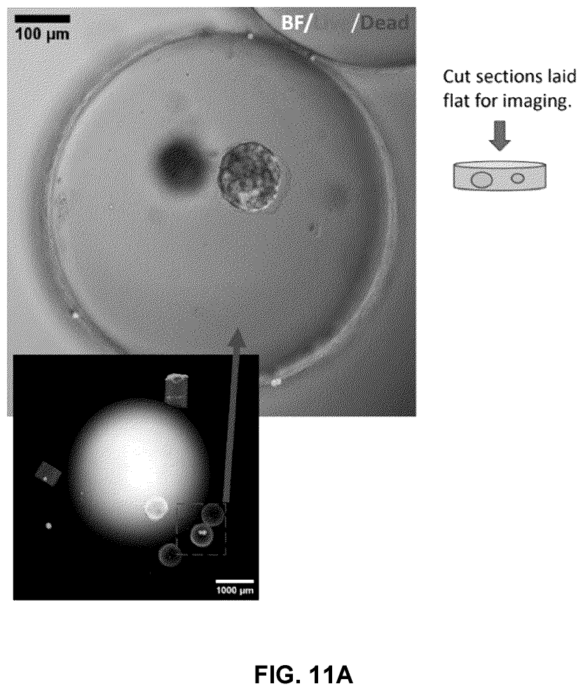

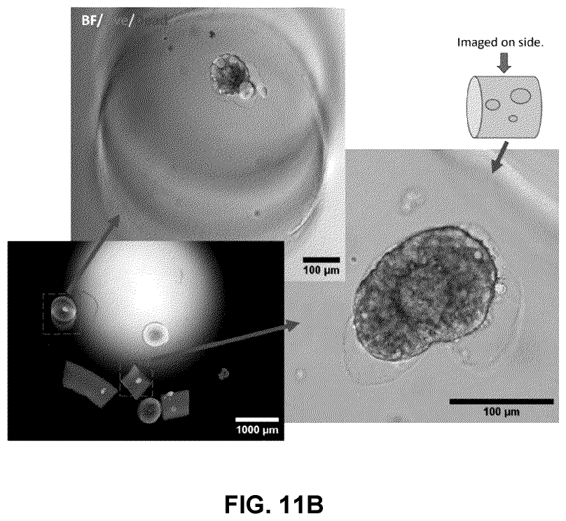

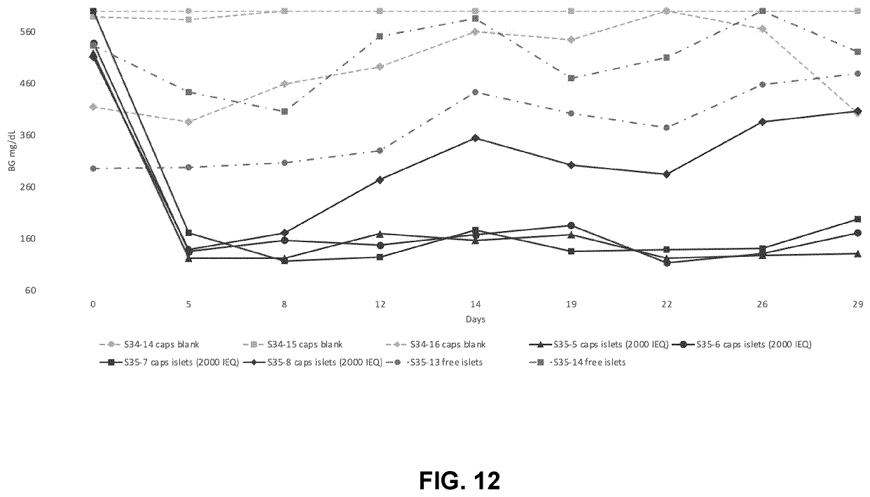

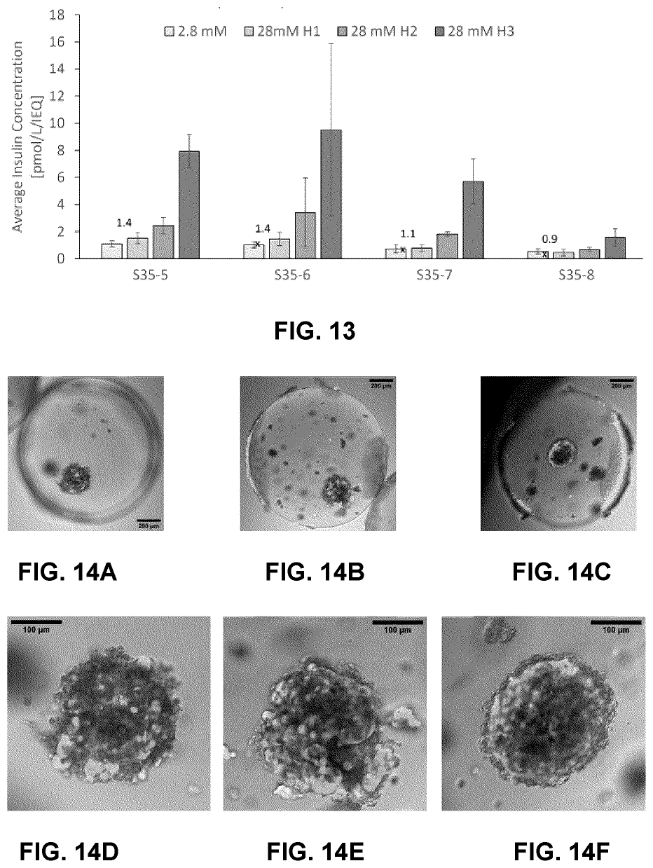

As shown in FIG. 8, after a short term (2 week) transplantation, explant GSIS—islets show function in two thirds of the rats (healthy rats receiving islets). FIGS. 9A-9B show the 2 week live/dead staining on explanted TEC-cy strings containing rat islets, implanted into healthy Wistar rats. Long term GSIS data (42 days) on retrieved explants is presented in FIGS. 10A-10B. A short term transplantation (14 days for conditions labeled Ahr-1, and Ahr-3) is compared to the long term (42 days) conditions labeled Ahr-4, Ahr-5 and Ahr-6. Imaging was also performed on the long term explants and is presented in FIGS. 11A-11B. BG data was tracked over the course of one month in diabetic mice implanted with TEC-cy strings at 3 islet loading densities (human islets) and TEC-cy (FIG. 12). As can be seen from FIG. 12 the implantation of encapsulated islets outperformed free islets as well as the blank controls. FIG. 13 shows the BG of diabetic mice treated with TEC-cy capsules with human islets after 29 days. FIGS. 14A-14F show images of live/dead staining of human islets in explanted TEC-cy capsules after 29 days in vivo in diabetic mice. As can be seen in FIGS. 14A-14F the islets remain viable.

Capsules that were explanted from the mice were assessed for pericapsular fibrotic overgrowth (PCO). All of the capsules that were retrieved from the explantation procedure were transferred to a microscope slide and imaged using a Nikon Eclipse Ti inverted microscope. The automated stage was used for software-controlled stage translation to capture brightfield images which were stitched to form a composite image using the Nikon NIS Elements AR 5.11.01 software. For PCO analysis, the individual capsules in the composite image were manually assessed according to the PCO scoring categories of 1, 2, 3, and 4, corresponding to overgrowth capsule coverage percentages of 0-24%, 25-49%, 50-74%, and 75-100%, respectively. It should be noted that the analysis is limited to assessing one side of the capsule.

### Formation of TEC-Cy Hydrogel Patches (Blank)

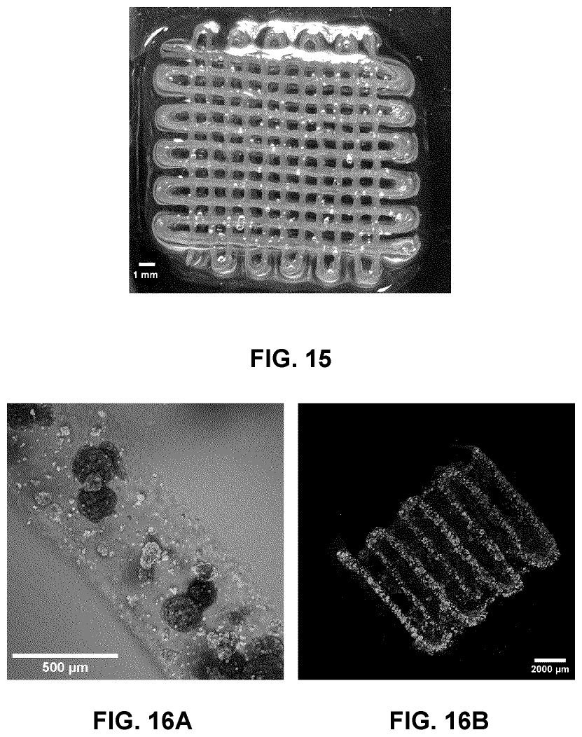

To prepare the TEC-cy hydrogel patches, the TEC-cy gel forming solution was prepared as described with 1-3 wt % alginate was loaded into a syringe for an Advanced Solutions BioBot Basic and fitted with a 0.5″ 20G blunt tip needle. FRESH™ (freeform reversible embedding of suspended hydrogels) support bath was prepared with 9 mM calcium chloride. FRESH™ baths act as a Bingham Plastic during printing, and the 9 mM calcium chloride effects partial ionic crosslinking of the alginate during printing, providing sufficient stiffness to the emerging string segments. Notably, this calcium concentration of 9 mM was low enough to allow further crosslinking of interconnecting layers within the ultimate three dimensional structure. Then, 10-15 mL of this FRESH™ support bath with 9 mM calcium chloride was placed in a single well of a 6-well plate. A predetermined patch shape was printed in a crosshatch pattern with inter-filament distances ranging from 250 to 1500 μm, and preferably 400 to 700 μm. The linear print speed was matched to the flow rate (which in turn is related to the applied pressure and the rheological characteristics of the bioink with this printer model) to obtain the targeted filament diameter. After the print was complete, the 6 well plate was placed in an incubator at 37° C. for 0.5-1 hour to allow the FRESH™ support bath to liquify completely. The patch was then removed and placed for fine minutes into a well of a 6 well plate with gelling bath consisting of 100 mM calcium chloride, 35 mM HEPES, and 0.45 wt. % sodium chloride at pH 7.4, before being rinsed with saline. In other instances, a barium gelling bath with 10 mM barium chloride, 35 mM HEPES, and 0.45 wt. % sodium chloride at pH 7.4 was used for 5 minutes. The obtained three-layer TEC-cy patch, formed with 3 wt. % alginate and cured with calcium chloride is shown in FIG. 15.

### Example 8: Encapsulation of Human Donor Islets in a TEC-cy Patch

For bioprinting patches with human donor islets, the TEC-cy gel forming solution was prepared as described, except the concentration of the polymers in HBS was 1.2 times greater and the amount of alginate used was 2.4 wt. %. The resulting solution was sterilized using 0.22 μm syringe filters in a BSC. The solution was carefully diluted with human donor islets in HBS in a 5:1 ratio to result in a printing solution that contains concentrations of polymers equal to the TEC-cy gel forming solution that was previously described with 2 wt % alginate content and >80 IEQ/cm of ultimate filament length of islets. This solution was loaded into a fitted syringe for an Advanced Solutions BioBot Basic placed in the same BSC and fitted with a 0.5″ 20G blunt tip needle. FRESH™ support bath was prepared in a sterile form by soaking in 70% Ethanol for 1 hour before purification via subsequently undergoing repeated rinses and centrifugation with 9 mM calcium chloride content. 10-15 mLs of this calcium chloride-laden FRESH™ support bath was placed in a single well 6 well plate. A four layer 2×2 cm patch was printed in a crosshatch pattern with inter-filament distances of 750 μm (albeit with offset layers). The print speed was matched with the flow rate (which is related to the applied pressure and the rheological characteristics of the bioink with this printer model) to obtain a filament diameter that matches what is desired for a given patch printing and resulted in a four layer patch in less than 3 minutes. Multiple of these patches were printed from the same ink and the resulting patches were cultured up to 4 days. Patches were selected on days 0, 1, 2, and 4, stained with Ethidium/Calcein AM live/dead stain, and assessed via confocal microscopy to observe the extent to which the islet viability was maintained over time. Patches were also assessed for their function via quantification of insulin release in response to glucose-stimulated insulin secretion (GSIS) experiments.

Confocal images of live/dead stained islet clusters in bioprinted TEC-cy patches with 2 wt. % alginate, show that islets can be printed at high concentrations (>80 IEQ/cm of filament length) while maintaining the viability of the clusters and keeping them away from the edges of the printed filaments over 7 days (FIGS. 16A-16B).

1. A hydrogel string comprising:
a thiol-ene crosslinked polymer comprising a first side-chain functionalized backbone polymer functionalized with an activated alkene crosslinked with a free or protected thiol-containing group present on a second side-chain functionalized backbone polymer; and
a biological material encapsulated within the thiol-ene crosslinked polymer.

2. The hydrogel string of claim 1, wherein the hydrogel string has an aspect ratio of at least 5.

3. The hydrogel string of claim 1, wherein the hydrogel string further comprises alginate, hyaluronic acid, gelatin, hydroxypropylcellulose, carboxymethylcellulose, methylcellulose, or elastin.

4. The hydrogel string of claim 1, wherein the biological material is a cell, a cell aggregate, or cell spheroid, and the hydrogel string optionally further encapsulates angiogenic and/or chemotactic agents.

5. The hydrogel string of claim 1, wherein the activated alkene is vinylsulfone, maleimide, acrylate or a methacrylate.

6. The hydrogel string of claim 1, wherein the thiol-containing group is 2-pyridinethiol or cystamine.

7. The hydrogel string of claim 1, wherein the backbone polymer is a homopolymer of polyacrylic acid, a homopolymer of polymethacrylic acid, or copolymers of acrylic acid and methacrylic acid.

8. The hydrogel string of claim 5, further comprising a capping agent on the surface of the hydrogel string neutralizing vinyl sulfone groups.

9. The hydrogel string of claim 1, wherein the hydrogel string has an outer diameter of less than 2000 μm.

10. The hydrogel string of claim 1, wherein the thiol-ene crosslinked polymer forms an outer shell encapsulating a core of the biological material.

11. The hydrogel string of claim 1, wherein the shell has a heterogeneous density, wherein an outer surface has a higher density than an inner surface as measured by fluorescence microscopy.

12. (canceled)

13. (canceled)

14. A process of producing a hydrogel string comprising:
continuously extruding or co-extruding a first polymer containing free or protected thiol groups and a second polymer containing vinyl groups into a bath, preferably an aqueous bath, containing a reactive agent to drive the gelation of the first and second polymers; and
allowing a crosslinking reaction between the thiol groups and the vinyl groups to occur.

15. (canceled)

16. The process of claim 14, wherein the extruding or co-extruding further comprises extruding or co-extruding a biocompatible polymer selected from the group consisting of alginate, hyaluronic acid, gelatin, hydroxypropylcellulose, carboxymethylcellulose, methylcellulose, or elastin with the first and second polymers.

17. The process of claim 14, wherein the biocompatible polymer is alginate and the bath comprises ions selected from the group consisting of calcium, barium, strontium, copper, zinc, manganese, cobalt, lead, iron, or aluminum.

18. The process of claim 14, wherein the reactive agent is a reducing agent, preferably tris (2-carboxyethyl) phosphine (TCEP) or tris (hydroxypropyl) phosphine (THPP).

19. The process of claim 14, further comprising providing a capping agent introduced prior to a final wash, to convert residual vinyl sulfone groups into more biocompatible groups.

20. The process of claim 14, wherein the ratio of thiol groups to vinyl (ene) groups is from 0.95:1.05 to 0.65:1.35.

21. The process of claim 14, comprising extruding or co-extruding the polymers into the bath through a blunt end syringe.

22. (canceled)

23. (canceled)

24. A method of transplanting a transplant comprising at least one hydrogel string as defined in claim 1, wherein the at least one hydrogel string is substantially retrievable from an implant site in a live mammalian body after a transplantation time of at least six weeks, the method comprising:
depositing the transplant onto a spread-out omental tissue of a subject in need thereof;
folding the omental tissue to form a pouch containing the transplant; and
securing the omental tissue in the pouch formation by suturing and/or using tissue glue, preferably wherein the tissue glue comprises a mixture of fibrinogen and thrombin, or analogues thereof.

25. The method of claim 24, wherein said method is an open surgery, or is a laparoscopic procedure.

26-39. (canceled)
